(19) United States
(12) Patent Application Publication
(10) Pub. No.: US 2026/0351389 A1
(43) Pub. Date: Nov. 5, 2026

(54) SYSTEMS AND METHODS FOR CONSOLIDATING, STREAMLINING, AND AUTOMATING THE SCHEDULING OF MEDICAL SERVICES, INSURANCE ELIGIBILITY, AND BENEFITS VERIFICATION, INSURANCE COVERAGE CONFIRMATION, COPAYMENT AND CLAIM PROCESSING IN REAL-TIME AT THE POINT-OF-SALE

(71) Applicant: Isaac Wright, Miami, FL (US)

(72) Inventor: Isaac Wright, Miami, FL (US)

(21) Appl. No.: 19/371,853

(22) Filed: Oct. 28, 2025

Related U.S. Application Data
(60) Provisional application No. 63/799,316, filed on May 2, 2025.

(51) Int. Cl. G16H 40/20; G06Q 40/08

(52) U.S. Cl. CPC G16H 40/20; G06Q 40/08; G06Q 40/084

(57) ABSTRACT

A software-based healthcare transaction system is provided comprising a Master Service Provider System (MSPS) that enables real-time scheduling, insurance verification, copayment processing, and claim submission. The MSPS includes modules for patient registration, provider onboarding, insurance company integration, credential management, and payment gateway communication. Patients submit insurance data and service preferences, which are verified through an external analytics system. Upon service completion, a one-time Service Completion Code (SCC) is generated and submitted to trigger claim generation. Claims are transmitted for adjudication, and providers may optionally receive immediate discounted payments. The system further supports post-payment compliance access for insurers based on configured permissions, and includes modules for non-emergency medical transportation registration, credential tracking, and user notifications.

### CROSS-REFERENCE TO RELATED APPLICATIONS

The present application claims priority to U.S. Provisional Patent Application 63/799,316 filed on May 2, 2025, entitled “A SOFTWARE SYSTEM AND METHOD FOR CONSOLIDATING, STREAMLINING AND AUTOMATING THE SCHEDULING OF MEDICAL SERVICES, INSURANCE ELIGIBILITY AND BENEFITS VERIFICATION, INSURANCE COVERAGE CONFIRMATION, COPAYMENT AND CLAIM PROCESSING IN REAL TIME AT POINT OF SALE” the entire disclosure of which is incorporated by reference herein.

### TECHNICAL FIELD

The present invention relates generally to healthcare management systems, and more particularly to software and methods for consolidating, streamlining, and automating the scheduling of medical appointments, verifying insurance eligibility and benefits, confirming insurance coverage, processing copayments, and executing claim submissions at the point of service.

### BACKGROUND

The field of healthcare administration has long been challenged by fragmented and disjointed workflows involving scheduling, insurance verification, copayment collection, and claims processing. Traditionally, these critical operations have been performed using a patchwork of legacy systems, manual procedures, and third-party service platforms, each with varying degrees of interoperability. This disconnected landscape has contributed to inefficiencies, increased operational costs, and a frustrating patient experience, particularly in the context of medical appointments and non-emergency medical transportation.

One of the core issues arises from the lack of centralized coordination among the various stakeholders involved in a patient's journey, patients, service providers, insurers, billing entities, and payment processors. Each party often operates within its own software environment, leading to repeated data entry, miscommunication, and delays. The verification of insurance eligibility and benefit coverage frequently requires separate interactions with insurers, which are often performed manually or through systems that provide limited transparency and real-time capabilities.

Compounding this problem is the delayed and cumbersome nature of healthcare payment and claims cycles. Providers commonly wait 30 to 90 days to receive reimbursement, during which time they must manage outstanding receivables and navigate frequent claim rejections due to coding inconsistencies or missing information. This delay undermines the financial stability of small practices and contributes to systemic administrative burden.

From the patient's perspective, uncertainty around insurance coverage, unexpected copayments, and last-minute appointment cancellations due to authorization failures are common pain points. These shortcomings erode patient trust, delay care, and create unnecessary complications for all parties involved. The inefficiencies are particularly pronounced in services involving transportation, where confirmation of eligibility and payment is essential before care can even be rendered.

Efforts to address these issues through electronic health records (EHRs), clearinghouses, and practice management software have only partially mitigated the problem. These solutions tend to address isolated functions, such as claims submission or patient intake, without offering a fully integrated, end-to-end process. As a result, healthcare providers still rely heavily on administrative staff to bridge the gaps between disparate systems and manually ensure operational continuity.

The need for a unified, automated solution that can centralize scheduling, verification, payment, and claim handling in a single, accessible interface has become increasingly clear. By closing the loop on these interconnected processes, the healthcare industry stands to gain substantial improvements in accuracy, efficiency, and patient satisfaction.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provide a software-based healthcare transaction system that consolidates, streamlines, and automates the scheduling, verification, payment, and claims processing functions associated with medical and transportation services. The core of the system is a Master Service Provider System (MSPS), a cloud-based software platform accessible via any internet-enabled device. The MSPS integrates a patient interface module, a service provider interface module, and an insurance company interface module, each designed to receive, store, and exchange information relevant to its user type. The MSPS further incorporates an Application Programming Interface (API) module, a payment gateway module, a claim submission module, and a compliance access module to facilitate real-time interactions among connected systems. The system operates in a secure environment and stores all transactional data in a structured and user-specific database. It is designed to eliminate the fragmentation that currently exists between disparate systems used by healthcare providers, payers, and patients.

The patient interface module within the MSPS enables a patient to register and input critical data necessary for the scheduling and billing of healthcare services. This data includes insurance policy information, appointment preferences, and multiple payment credentials, such as credit cards, debit cards, ACH accounts, digital wallets, and prepaid vouchers. The module also provides a user dashboard where patients can view upcoming appointments, verify benefit coverage, and select service providers based on cost or location. The patient may initiate scheduling for medical treatment or non-emergency medical transportation (NEMT) services directly through this interface. The system supports encrypted storage of all submitted information and restricts unauthorized access through multi-factor authentication. This module thus serves as the entry point for patient participation in the healthcare service lifecycle within the MSPS.

The service provider interface module within the MSPS allows service providers to create and manage their accounts, upload verification documents, and define service types, pricing, and scheduling availability. Service providers may include individual practitioners, transportation companies, clinics, hospitals, and assisted living facilities. The module supports uploading of credentials such as licenses, CPR training certificates, driving histories, insurance certificates, and equipment records. Providers may also register vehicles and staff within the system, assigning each a unique identifier for transaction tracking purposes. Additionally, service providers can view appointment requests, respond to scheduling notifications, and receive real-time updates on payment or claim statuses. All such interactions are stored within the MSPS for audit and compliance tracking.

The insurance company interface module is configured to support the registration of private and public insurers, including but not limited to Medicaid and Medicare providers. The module allows insurance companies to input payer IDs, plan types (e.g., HMO, PPO, POS, EPO), NPI numbers, and contact details. Once registered, insurance companies may access policy validation tools, track submitted claims, and review compliance-related documents linked to specific services they fund. All data entered by the insurance company is mapped to its unique identifiers for accurate integration during claim processing. The MSPS ensures secure access for these stakeholders by validating credentials and limiting access to claim-relevant data only. This module allows for seamless integration of the payer into the broader transactional network governed by the MSPS.

The MSPS includes an Application Programming Interface (API) module that facilitates data exchange between the MSPS and a Healthcare Software/Analytics Company (HSAC) system. In some embodiments, the HSAC system can be the system of a third-party provider or a system of the patient's insurance company. The API is configured to transmit a patient's insurance policy information to the HSAC system and request verification of eligibility and benefit coverage. The HSAC responds with a structured response containing the applicable coverage details, which the MSPS uses to confirm whether the requested service is authorized under the policy. This interaction occurs in real time, providing immediate feedback to the patient and service provider. If benefits are denied, the system alerts the patient and provides options to proceed using alternate payment methods. The API module ensures standardized, secure, and efficient communication between disparate systems that would otherwise require manual reconciliation.

Upon receiving eligibility confirmation from the HSAC system, the MSPS presents the patient with an appointment confirmation page that includes all relevant service data, including copayment obligations. The MSPS includes a payment gateway module configured to process such copayments using the stored payment credentials provided by the patient. This gateway supports a variety of payment formats, including but not limited to debit/credit cards, ACH transfers, QR code scanning, digital wallets, and installment payments. Once a copayment is successfully processed, the appointment is finalized, and all data is logged for future billing or audit purposes. The MSPS sends notification to the service provider to prepare for service delivery. This integrated flow ensures the entire scheduling and payment process can be completed within a single session by the patient.

After the service has been rendered, the MSPS generates a one-time-use Service Completion Code (SCC) and transmits it to the patient's device via email or SMS. The SCC is a unique alphanumeric string that functions as proof that the service was completed. The patient is required to provide this SCC to the service provider, who then inputs it into the MSPS via their provider interface module. This action triggers the MSPS's claim submission module, which gathers all associated service codes, patient insurance data, and provider credentials to generate a structured insurance claim. The claim is immediately transmitted to the HSAC system for processing. The use of the SCC guarantees that services are billed only when verified by the patient, thereby reducing fraud and disputes.

The MSPS's claim submission module is tightly integrated with both the provider records and the API module. Upon receiving the SCC from the provider, the MSPS cross-references stored data, such as CPT, STC, ICD-11, and HCPCS codes associated with the treatment, along with claim adjustment group codes if applicable. The claim is formatted and sent to the HSAC system, which responds with an acceptance or rejection status. If the claim is rejected, the MSPS notifies the provider and patient, offering options for manual review or appeal. If accepted, the system updates all relevant account dashboards and stores the claim history for future reference. This real-time submission and feedback loop drastically reduces billing cycles and improves transparency for all involved parties.

An innovative feature of the MSPS is its ability to issue a discounted immediate payment to the service provider once a claim has been successfully submitted. This feature is managed by the payment gateway module and allows the provider to bypass traditional 30-90 day reimbursement windows. If selected, the MSPS calculates a discounted amount based on a pre-configured fee structure and disburses funds to the provider's registered account. This optionality supports financial flexibility for providers and increases adoption of the MSPS platform. The original claim remains open with the insurance company, and any variance in reimbursement is settled between the MSPS and the payer. The system ensures that financial incentives align with operational efficiency and provider satisfaction.

The MSPS includes a compliance access module designed to facilitate regulatory oversight and reporting for insurance companies. Upon successful claim processing and payment, this module allows the insurance company associated with the transaction to access specific data relevant to the service. This may include provider credentials, patient age or gender, service type, diagnosis codes, and regulatory documents. Access is governed by permissions established by the patient and the provider and may be limited to specific data elements. Insurance companies may view charts, graphs, and exportable reports summarizing services rendered under their coverage plans. This feature improves transparency, reduces fraud, and assists insurers in fulfilling their oversight obligations.

A CPT/STC/ICD-11/HCPCS code-matching engine is integrated within the MSPS and serves to accurately assign medical and procedural codes to each transaction. This engine allows the provider or patient to select from pre-configured service menus, automatically generating the appropriate billing codes without requiring extensive manual input. It also retrieves related claim adjustment group codes and provides warnings in the event of coding inconsistencies. The MSPS uses this engine to ensure that claims submitted to the HSAC system are accurate, properly formatted, and compliant with industry standards. Code matching is executed during both the scheduling and submission phases to prevent discrepancies. The result is a measurable decrease in claim rejections and administrative burden.

To facilitate access to nearby service providers, the patient interface module includes a geographic service locator. This component identifies the patient's real-time location and filters available providers within a specified radius. The system cross-references provider availability, service types, and price structures to generate a personalized shortlist. Patients can sort results by cost, provider ratings, or distance and select a preferred provider directly within the MSPS. Once a provider is selected, scheduling proceeds with eligibility verification and payment steps as described above. This feature enhances usability and supports faster, more informed decision-making by patients.

In scenarios where insurance eligibility is denied, the MSPS is configured to present the patient with alternative payment options. This functionality is built into the payment gateway module and supports immediate transition from insurance-based payment to out-of-pocket methods. Patients may use stored financial instruments or select third-party financing options also integrated into the platform. The system recalculates service costs in real time based on the selected method and updates provider notifications accordingly. The SCC-based confirmation process remains the same, ensuring all completed services are still verified before billing. This fallback pathway ensures services are not denied solely due to coverage issues.

All transaction data within the MSPS is stored in a structured, user-specific database that supports historical queries, regulatory audits, and personalized reporting. Each user—patient, provider, or insurer—can access their own transactional history through secured dashboards. These records include timestamps, payment confirmations, claim status, service notes, and downloadable receipts. The system supports exporting in standard formats such as PDF and CSV for off-platform review. Data integrity is maintained using redundancy and encryption protocols. This persistent recordkeeping capability distinguishes the MSPS from legacy scheduling or billing systems.

The MSPS is uniquely designed to accommodate scheduling and billing for non-emergency medical transportation services in addition to traditional medical treatments. During registration, the patient may specify transportation needs such as wheelchair, stretcher, or ambulatory assistance. The MSPS filters transportation providers based on equipment and vehicle type and presents only compliant providers for selection. The same insurance verification, SCC issuance, and claim processing flow applies. Transportation providers register vehicles and personnel under their provider account and upload compliance documents like licenses and vehicle insurance. This feature bridges a common gap in the medical access ecosystem by addressing transportation logistics and payment.

The MSPS includes robust features for service provider account setup, allowing both individual and institutional entities to register their workforce, vehicles, and service offerings. For example, transportation companies may upload vehicle details such as make, model, and year, and register drivers by providing licenses, background checks, and drug screening results. Medical professionals may input education, license numbers, and NPI credentials. The system assigns unique identifiers to each provider profile, vehicle, and user, allowing traceability throughout the service lifecycle. These identifiers are used during claim generation and compliance checks to ensure all elements of the service are accurately linked and recorded. This ensures full regulatory compliance for each rendered service and aids insurance companies during audits or investigations.

In the context of medical treatment, the MSPS enables patients to search for available providers using keywords or identifiers like Payer ID or NPI number. Patients can browse available appointment times and provider profiles before selecting a treatment slot. The MSPS collects insurance policy information and submits it to the HSAC system via API for benefit validation. Once validated, the MSPS calculates the applicable copay and presents the total cost of treatment. Patients then confirm and process the copay using stored payment methods. This end-to-end workflow is completed without requiring telephone calls, manual forms, or third-party scheduling systems, resulting in a significantly improved patient experience.

The MSPS supports multiple user journeys, including scheduling initiated by the patient, the provider, or a medical facility on behalf of the patient. When a medical facility or provider schedules an appointment, they input the patient's insurance and demographic information directly into the MSPS. The same verification and copayment workflow is executed on behalf of the patient, and the appointment is logged under both user accounts. The SCC is generated post-appointment and delivered to the patient, who must validate the service by returning the code to the provider. This collaborative approach allows providers to manage complex schedules while maintaining the integrity of the transaction flow. Such flexibility is essential for larger facilities and institutions serving patients with limited digital access.

The transaction workflow culminates in the submission of the SCC by the service provider, triggering the claim submission module to transmit all relevant codes and metadata to the HSAC system. The HSAC system evaluates the claim, and the MSPS receives a decision in real time. The service provider is then presented with a choice: receive immediate payment at a discount or await standard reimbursement from the insurer. This dual-option pathway allows providers to manage their own financial strategy based on current cash flow needs. The MSPS executes disbursement automatically upon selection and updates the provider's account dashboard accordingly. This integrated real-time transaction logic creates a seamless bridge between care delivery and financial closure.

Following service completion and payment processing, the MSPS activates the compliance access module for the insurance company associated with the transaction. This module allows authorized representatives to review detailed records, including provider credentials, service codes, patient demographics (as permitted), and outcome data. Patients and providers may opt to enable or restrict data visibility according to privacy preferences or regulatory requirements. The insurance company may use the data for trend analysis, fraud detection, or reimbursement reconciliation. These features establish the MSPS as not only a transactional hub, but also a compliance and oversight platform. The invention thus addresses the full lifecycle of healthcare scheduling, payment, and compliance from a unified, automated architecture.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a computer system architecture, according to some embodiments;

FIG. 2 is a system architecture diagram illustrating the Master Service Provider System (MSPS) in communication with a service provider device, patient device, insurance company device, payment gateway system, and healthcare software/analytics company system over a network, according to some embodiments;

FIG. 3 is a block diagram of an application program executed by the MSPS, comprising communication, display, registration, payment, claim processing, and compliance control modules, according to some embodiments;

FIG. 4 is a flowchart illustrating a method for scheduling a medical or transportation service, verifying insurance coverage, and confirming copayment through the MSPS, according to some embodiments;

FIG. 5 is a flowchart illustrating the generation, transmission, and verification of a Service Completion Code (SCC) to initiate claim processing, according to some embodiments;

FIG. 6 is a flowchart depicting the construction and submission of a healthcare claim from the MSPS to a healthcare software/analytics company system, according to some embodiments;

FIG. 7 is a flowchart showing the provider's option to receive immediate discounted payment through the payment gateway system following claim submission, according to some embodiments;

FIG. 8 is a flowchart illustrating permission-based post-payment access to service and credential data by an insurance company, according to some embodiments;

FIG. 9 is a flowchart showing the registration of non-emergency medical transportation (NEMT) vehicles and drivers within the MSPS, according to some embodiments;

FIG. 10 is a flowchart illustrating the upload, indexing, and compliance management of provider credential documents, according to some embodiments;

FIG. 11 is a flowchart depicting the calculation, authorization, and processing of patient copayments via the payment gateway system, according to some embodiments;

FIG. 12 is a flowchart showing the notification delivery workflow for communicating scheduling, payment, and claim status events to system users, according to some embodiments;

FIG. 13 illustrates a screenshot of the transportation company onboarding interface displaying business certificate, license, and insurance upload fields, according to some embodiments;

FIG. 14 illustrates a screenshot of the vehicle registration interface enabling entry of vehicle details including model, manufacture year, and VIN number, according to some embodiments;

FIG. 15 illustrates a screenshot of the pricing configuration interface for inputting insurance and private transport rates, according to some embodiments;

FIG. 16 illustrates a screenshot of the driver credential upload interface allowing submission of licenses, certifications, and background checks, according to some embodiments;

FIG. 17 illustrates a screenshot of the fleet management interface showing a live vehicle map and driver assignment controls, according to some embodiments;

FIG. 18 illustrates a screenshot of the job offer interface enabling drivers to accept or reject transport assignments, according to some embodiments;

FIG. 19 illustrates a screenshot of the navigation interface displaying routing to the patient pickup location, according to some embodiments;

FIG. 20 illustrates a screenshot of the vehicle and insurance document upload interface for transportation providers, according to some embodiments;

FIG. 21 illustrates a screenshot of the insurance and private pricing configuration interface for ambulance and transport services, according to some embodiments;

FIG. 22 illustrates a screenshot of the driver document upload interface for CDL, CPR certification, and background verification, according to some embodiments;

FIG. 23 illustrates a screenshot of the fleet dashboard interface showing available vehicles and driver assignment options, according to some embodiments;

FIG. 24 illustrates a screenshot of a transport request interface displaying trip details and driver acceptance options, according to some embodiments;

FIG. 25 illustrates a screenshot of the en-route navigation interface guiding the driver to the pickup location, according to some embodiments;

FIG. 26 illustrates a screenshot of the en-route navigation interface guiding the driver to the drop-off location, according to some embodiments;

FIG. 27 illustrates a screenshot of the payout interface displaying ride completion details and immediate or delayed payment options, according to some embodiments;

FIG. 28 illustrates a screenshot of the insurance company transaction search interface for viewing transport and appointment records, according to some embodiments;

FIG. 29 illustrates a screenshot of the insurance company analytics interface displaying transport usage and distance metrics, according to some embodiments; and

FIG. 30 illustrates a screenshot of the insurance payment and claim settlement interface for processing reimbursements, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a software-based healthcare transaction system designed to consolidate, streamline, and automate critical functions associated with the delivery of healthcare and transportation services. These functions include scheduling, insurance eligibility verification, payment processing, and real-time claim submission. The system comprises a Master Service Provider System (MSPS), which serves as the central software platform through which all users interact. The MSPS is configured to operate on a cloud-based server and is accessible via any internet-enabled device, such as a smartphone, tablet, or computer. Through a modular architecture, the MSPS allows patients, service providers, insurance companies, healthcare software/analytics companies, and payment gateways to communicate and transact within a single integrated environment.

The MSPS includes a patient interface module that facilitates the registration of patients and enables patients to provide and manage their insurance policy information, appointment preferences, and payment credentials. The patient interface module presents a user-friendly dashboard that allows patients to browse service options, initiate scheduling requests, and receive appointment confirmations. The information input by the patient is securely stored and is used to facilitate subsequent interactions involving insurance verification, copayment processing, and compliance reporting. The patient interface module may also include real-time location tracking and filtering tools to match patients with nearby service providers.

The MSPS further includes a service provider interface module configured to enable the registration of service providers and the receipt of service credentials, regulatory documents, pricing information, and availability schedules. Service providers may include medical professionals, transportation operators, medical facilities, hospitals, and assisted living entities. Upon registration, each service provider is assigned a unique identifier. Providers can use the interface to manage their schedules, respond to patient requests, input service data, and receive updates related to insurance claims and payments. The service provider interface module enables full operational control over the provider's interaction with the MSPS platform.

An insurance company interface module is also included within the MSPS to enable insurance companies to register with the system and submit payer ID data, plan types, and network participation information. The insurance company interface module facilitates the exchange of compliance data and claim information between the MSPS and the insurance company. Registered insurance companies may view covered service histories, access regulatory documents, and manage plan data associated with claims processed through the MSPS. Permissions and access to data are governed by the compliance access module, discussed in further detail below.

The MSPS includes an Application Programming Interface (API) module configured to securely transmit insurance policy information collected through the patient interface module to a Healthcare Software/Analytics Company (HSAC) system. The API module is further configured to receive responses from the HSAC system indicating benefit verification status and insurance eligibility. These responses are used by the MSPS to confirm or deny patient eligibility in real time. The API module ensures that sensitive patient and policy data is transmitted in compliance with applicable data privacy standards, and it serves as the primary conduit for initiating insurance-related validations.

The MSPS includes a payment gateway module configured to process copayments and alternative payments using stored payment methods within the patient interface module. The payment gateway module supports a variety of transaction types including debit cards, credit cards, ACH transfers, digital wallets, mobile payments, prepaid vouchers, QR codes, and cryptocurrency wallets. Patients may register one or more payment methods upon account creation, and the system can prioritize or suggest payment methods based on availability or past usage. The payment gateway module ensures secure, encrypted processing of all transactions and logs payment details in the MSPS database.

The system further comprises a claim submission module configured to automatically generate and transmit a healthcare claim upon receipt of a Service Completion Code (SCC). The SCC is a one-time-use alphanumeric identifier issued by the MSPS to the patient after the service provider marks a service as completed. The patient receives the SCC via SMS, email, or app notification and is required to submit it to the service provider. Once the SCC is submitted into the MSPS, the claim submission module activates and assembles all necessary data including service codes, provider credentials, and patient information to generate a complete insurance claim. The claim is then transmitted to the HSAC system for real-time processing.

A compliance access module is integrated within the MSPS to enable insurance companies to access relevant service, billing, and regulatory data following a completed and paid-for healthcare transaction. The compliance access module is governed by permission rules set by the patient and service provider during registration or transaction execution. The data made available may include provider credentials, service codes, patient demographics (where permitted), and documentation supporting medical necessity or provider qualification. The compliance access module allows insurers to conduct audits, verify policy compliance, and evaluate service history with minimal administrative overhead.

The MSPS further includes a CPT/STC/ICD-11/HCPCS code-matching engine configured to associate medical services and procedures with standardized billing codes. The code-matching engine is accessible through both the patient interface module and the service provider interface module. When a patient selects a service type or a provider inputs a treatment procedure, the code-matching engine automatically populates the applicable billing codes, including associated claim adjustment group codes where applicable. This ensures consistent claim formatting and reduces the incidence of insurance rejections due to incorrect or missing codes. The code-matching engine operates in real time and updates dynamically based on user input and MSPS database logic.

To enhance accessibility and efficiency, the patient interface module includes a geographic service locator configured to match patients with available service providers based on GPS location data. Patients may specify a search radius, transportation needs, and service type to generate a tailored list of potential providers. The MSPS uses scheduling and compliance data from the service provider interface module to ensure that only compliant and available providers are displayed. This feature streamlines patient decision-making and eliminates the need for third-party transportation booking or medical scheduling applications. All selected provider data is used in subsequent insurance verification and claim processing steps.

In the event that insurance eligibility is denied, or benefits are insufficient to cover the service, the MSPS enables the patient to select from alternative payment methods. The payment gateway module presents these options within the patient interface module, allowing the user to authorize payment from stored cards, bank accounts, or third-party financing tools. The MSPS recalculates the payment obligation and confirms the appointment once the alternative payment is authorized. This fallback mechanism ensures that services are not delayed or denied due to eligibility failures and provides patients with real-time financial control over their care decisions.

The MSPS maintains a persistent record of all transactions in a user-specific, encrypted database. This includes scheduling data, insurance policy interactions, billing codes, payment confirmations, SCC issuance logs, and claim submission statuses. Patients, service providers, and insurance companies may access their respective histories through secured dashboards. The MSPS database supports export of data in standard formats such as PDF or CSV, and may be used for compliance audits, reporting, or dispute resolution. The persistent nature of this recordkeeping ensures data traceability across the lifecycle of every transaction.

The MSPS supports scheduling for non-emergency medical transportation services as well as traditional medical treatments. Patients may specify whether the requested service involves mobility aids such as wheelchairs or stretchers. Service providers in the transportation category register vehicles, drivers, and equipment under their accounts. The MSPS then matches these resources with patient transportation needs. The system uses the same API, SCC, and claim submission infrastructure to support transportation claims. This integration addresses a critical gap in healthcare access, particularly for patients requiring assisted travel.

The MSPS includes a scheduling and availability engine within both the patient and provider interface modules. Providers may pre-configure blackout dates, hours of operation, and service types available on specific days. Patients select available slots based on real-time availability, and the MSPS locks in appointment times once verification and payment are complete. This capability ensures that schedules are synchronized and reduces the need for manual coordination. The scheduling engine integrates with the SCC and claim submission modules to ensure each appointment results in a verifiable and billable transaction.

All components of the MSPS, including the patient interface module, service provider interface module, insurance company interface module, Application Programming Interface (API) module, payment gateway module, claim submission module, compliance access module, and CPT/STC/ICD-11/HCPCS code-matching engine, work together to create a unified and automated healthcare transaction environment. This architecture ensures data consistency, reduces administrative burden, and improves transparency for all stakeholders. The MSPS replaces the fragmented multi-platform approach currently used in healthcare scheduling and billing with a cohesive and real-time system. It enables patients to receive timely care, providers to be paid promptly, and insurers to verify compliance and reduce fraud. The present invention thus addresses a long-felt but unresolved need in the healthcare transaction ecosystem.

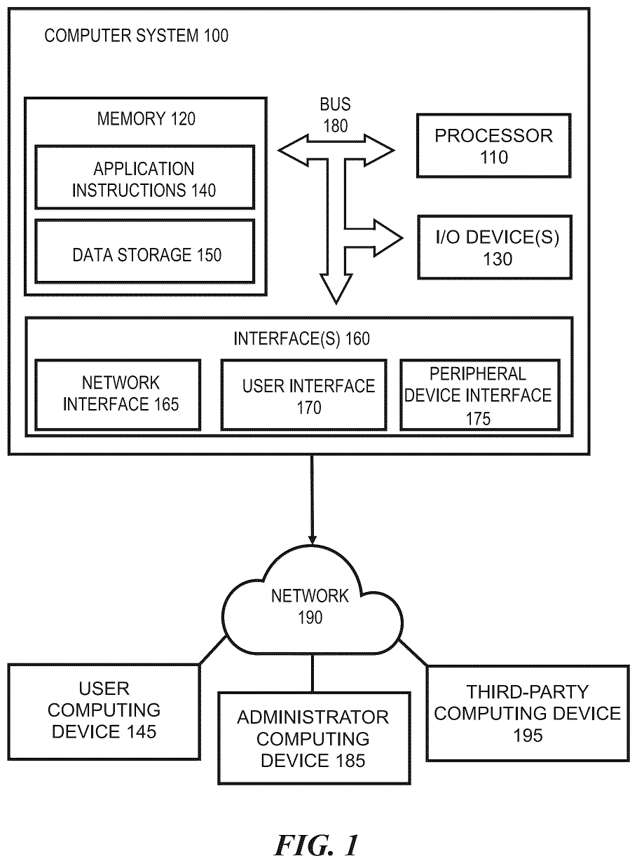

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, Swift, Python, C, C++, C #, JAVA, JAVASCRIPT, PERL, Kotlin, Dart, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. Hosting platforms may be implemented including public or private clouds. Further, various cloud storage system including table storage, blob storage databases, etc. may be used. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

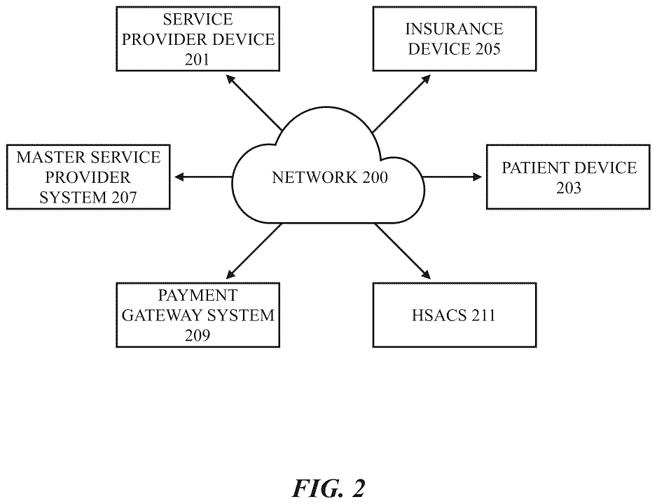

FIG. 2 illustrates a system-level overview of the communication architecture supporting the software-based healthcare transaction system, as executed via the Master Service Provider System (MSPS). The MSPS is configured to operate within a network 200, which includes internet-based, cellular, and cloud-enabled infrastructure. The network 200 enables data exchange between the MSPS and a plurality of external components, each serving a defined role within the healthcare transaction lifecycle. These components include a Service Provider Device (SPD) 201, a Patient Device (PD) 203, an Insurance Company Device (ICD) 205, a Master Service Provider System (MSPS) 207, a Payment Gateway System (PGS) 209, and a Healthcare Software/Analytics Company System (HSACS) 211.

The network 200 represents the communication medium through which the MSPS and all participating entities securely exchange data. In various embodiments, the network 200 may comprise one or more of the following: the Internet, wireless communication protocols (e.g., 4G, 5G, LTE), secure LAN or WAN connections, VPN tunnels, or cloud service frameworks. The network 200 is configured to support secure and encrypted transmission protocols, ensuring the integrity and confidentiality of patient data, insurance information, payment credentials, and claim documentation exchanged among the connected systems.

The Service Provider Device (SPD) 201 is an internet-capable computing device operated by a service provider. In various embodiments, the SPD 201 may comprise a desktop computer, laptop, tablet, or mobile device, and may be used by individual practitioners, medical facilities, transportation companies, or administrative staff. The SPD 201 is configured to communicate with the MSPS 207 over the network 200 to perform registration, receive scheduling requests, confirm appointments, input service completion notifications, and submit Service Completion Codes (SCCs). The SPD 201 also receives billing feedback and claim status updates from the MSPS.

The Patient Device (PD) 203 is a user-facing device configured to enable a patient to access and interact with the MSPS 207. The PD 203 may be a smartphone, tablet, laptop, or any other internet-enabled device capable of executing secure web or mobile applications. Through the PD 203, the patient registers for an account, inputs insurance information, selects payment methods, searches for service providers, and initiates appointment scheduling. The PD 203 also receives notifications related to eligibility verification, copayment responsibilities, appointment confirmations, and SCCs following service completion.

The Insurance Company Device (ICD) 205 represents the digital interface through which an insurance company accesses the MSPS 207. The ICD 205 may be used by both private and public insurers, including government entities such as Medicare and Medicaid. The ICD 205 is configured to allow the insurer to register policy information, update payer IDs, verify the status of claims, and review compliance documentation. The ICD 205 also serves as the access point through which the insurance company receives regulated, permissioned views into service data, such as provider credentials, patient demographics, and treatment details.

The Master Service Provider System (MSPS) 207 is the central software platform that orchestrates all interactions between the patient, service provider, insurance company, payment gateway, and healthcare analytics company. The MSPS 207 comprises a modular architecture that includes multiple subcomponents: a registration engine, a scheduling engine, an eligibility verification module, a payment gateway interface, a claim generation and submission module, and a compliance reporting engine. The MSPS 207 receives, processes, and routes data received from SPD 201, PD 203, ICD 205, HSACS 211, and PGS 209 in real time.

The MSPS 207 stores and interprets standardized billing and medical classification codes, including CPT, STC, ICD-11, HCPCS, Place of Service codes, and Claim Adjustment Group codes. These codes are used by the MSPS 207 to generate accurate insurance claims, ensure billing compliance, and calculate copayment responsibilities. The MSPS 207 also includes an Application Programming Interface (API) that enables secure communication with external systems, such as the HSACS 211, for benefit verification and claim adjudication.

The Payment Gateway System (PGS) 209 is an external digital infrastructure that processes financial transactions initiated within the MSPS 207. The PGS 209 is configured to handle a wide range of payment types including credit cards, debit cards, ACH transfers, digital wallets, prepaid vouchers, and installment payments. When the MSPS 207 determines that a copayment is required, it invokes the PGS 209 to execute the transaction based on the patient's selected or default payment method. The PGS 209 also supports real-time disbursements to service providers in the event that an immediate discounted payment is selected following successful claim submission.

The Healthcare Software/Analytics Company System (HSACS) 211 is a third-party system that performs real-time insurance eligibility and benefit verification and claim adjudication functions. The MSPS 207 transmits insurance policy data and billing codes to the HSACS 211 via a secure API over the network 200. In response, the HSACS 211 returns verification results including policy status, covered services, copayment amounts, and benefit restrictions. After a service is completed and an SCC is submitted by the service provider, the MSPS 207 transmits a structured claim file to the HSACS 211, which processes the claim and returns a confirmation or denial.

Each of the devices—SPD 201, PD 203, ICD 205—communicates directly with the MSPS 207, but not necessarily with one another. The MSPS 207 acts as the centralized hub that synchronizes all interactions, ensuring proper sequencing of events such as account creation, insurance verification, payment clearance, and compliance auditing. In this architecture, the MSPS 207 eliminates the need for participants to manually bridge between systems, thereby automating processes that traditionally require administrative oversight.

The flow of data from PD 203 to MSPS 207 typically begins with the patient initiating a request to schedule a service. The MSPS 207 then evaluates the request, queries the HSACS 211 for insurance eligibility, and calculates any necessary copayment. If coverage is confirmed, the MSPS 207 requests a payment authorization from the PGS 209 and notifies both the PD 203 and SPD 201 of appointment confirmation. Upon service completion, the SPD 201 inputs the SCC, which prompts MSPS 207 to submit a claim to HSACS 211 and optionally disburse a payment via PGS 209.

The ICD 205 interacts with the MSPS 207 at the end of the service lifecycle, primarily for compliance and audit purposes. Once the insurance company is identified as the payor for a given transaction, the MSPS 207 associates that transaction with the ICD 205's unique identifiers (e.g., NPI, EIN, payer ID). The MSPS 207 then makes available a restricted data set—including treatment details, provider credentials, and service compliance logs—subject to the access rules configured by the patient and provider.

The SPD 201 and PD 203 rely on real-time synchronization with the MSPS 207 for operational efficiency. For instance, if a service provider updates availability or costs, this information is immediately reflected in the patient's appointment search interface. Similarly, notifications regarding copay requirements, SCC issuance, and payment receipts are dispatched in real time to both parties via the MSPS 207. This synchronous architecture ensures a frictionless and transparent experience for both patients and providers.

The HSACS 211 and PGS 209 are designed to operate autonomously with the MSPS 207 using standardized interfaces and data formats. The MSPS 207 includes formatting engines to translate internal data into claim-compliant structures accepted by HSACS 211, and payment request messages compatible with PGS 209. These integrations reduce latency and error rates in the claim and payment cycles, further reinforcing the automation and scalability of the system.

In summary, FIG. 2 demonstrates the comprehensive connectivity and real-time data orchestration capabilities of the MSPS 207 within network 200. Each device and system in communication with the MSPS 207 serves a distinct function, but all are synchronized via the MSPS's unified platform. The design eliminates fragmentation in the healthcare scheduling, billing, and payment pipeline by introducing a hub-and-spoke model where the MSPS 207 serves as the core transaction orchestrator. This architecture supports automated workflows, improves compliance, and dramatically shortens the revenue cycle in both medical treatment and non-emergency medical transportation scenarios.

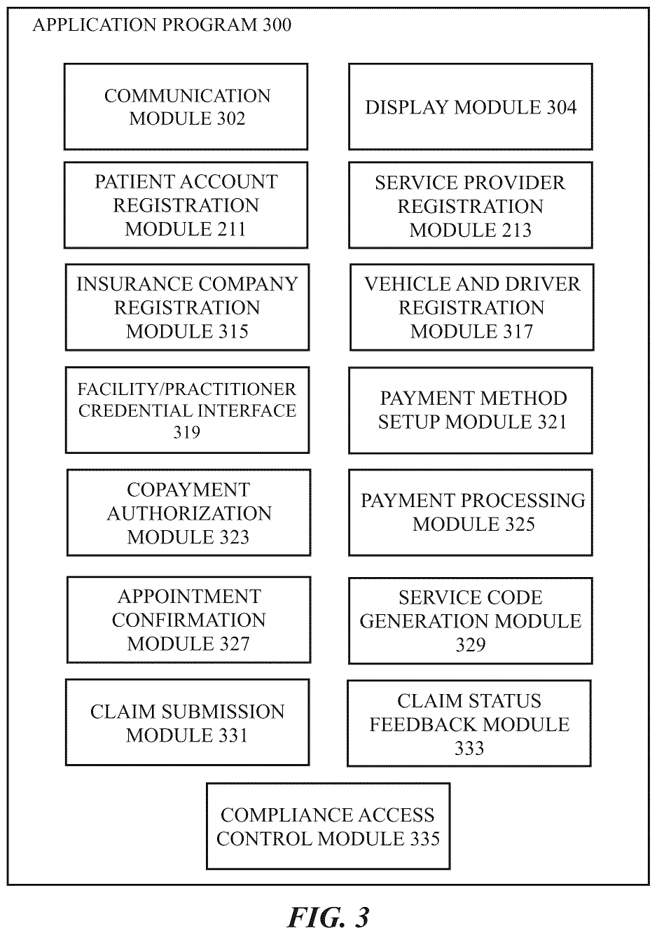

FIG. 3 is a block diagram illustrating the modular software architecture of the application program 300 executed by the Master Service Provider System (MSPS). The application program 300 comprises a plurality of functional modules, each configured to perform a specific role within the healthcare transaction lifecycle. These modules work in concert to facilitate account registration, insurance verification, appointment scheduling, payment processing, claim generation, and regulatory compliance. The modular design allows the system to dynamically adapt to user role types (e.g., patient, service provider, insurer) and ensures that each transaction is routed through secure, validated workflows. Communication among the modules is managed internally by the application program 300 and externally through secure connections to third-party systems, including payment gateways and healthcare analytics platforms. The architecture promotes scalability, maintainability, and regulatory transparency while enabling real-time automation of healthcare operations across a diverse network of stakeholders.

The application program 300 forms the central logic engine of the Master Service Provider System (MSPS). It is configured to manage and orchestrate a variety of interconnected software modules, each of which contributes to the system's goal of automating scheduling, billing, insurance verification, payment, and compliance. The application program 300 may operate in a cloud-hosted environment, ensuring high availability, scalability, and data redundancy across diverse geographical regions. It interacts with both external user devices and third-party systems in real time to facilitate seamless, end-to-end healthcare transactions. All user actions and module-level functions are routed through the application program 300 to ensure data consistency, security, and regulatory compliance. The system architecture may support modular upgrades and dynamic loading of features based on user needs or account types.

In operation, the application program 300 serves as the orchestration layer through which information flows from patients, service providers, and insurers to external systems such as payment gateways and healthcare analytics services. The modular framework allows the application to handle complex workflows involving registration, scheduling, insurance eligibility determination, and payment processing in a flexible and secure manner. Administrative control, error handling, and user permissions are also managed centrally within this component. The application program 300 maintains logs of all operations and integrates with auditing and reporting tools to ensure legal and operational traceability.

The communication module 302 is responsible for establishing and maintaining data exchanges between the MSPS and all external or internal endpoints. This includes communication with patient devices, service provider devices, insurance company systems, payment gateways, and healthcare software/analytics systems. The communication module 302 utilizes secure protocols such as HTTPS, SSL/TLS, and API-based encryption to transmit sensitive information like insurance policy details, billing codes, and payment credentials. Its design ensures that real-time interactions are synchronized and verified for integrity, ensuring patient safety and regulatory compliance. The module supports both synchronous and asynchronous communication workflows depending on the criticality and nature of the data being exchanged.

In certain embodiments, the communication module 302 may include failover systems and retry logic to ensure message delivery even during network instability. It works closely with other modules, such as the copayment authorization module and claim submission module, to provide reliable transmission of mission-critical data. Furthermore, it may provide real-time status indicators for users—such as confirmation messages, alerts, and transaction receipts—ensuring transparency and user confidence throughout the process. Its logging functionality assists in debugging, security analysis, and compliance reporting.

The display module 304 manages the graphical user interface (GUI) components that are presented to users interacting with the MSPS. These users may include patients, service providers, insurance company representatives, or administrative staff. The module is designed to adapt its interface based on user role, device type, and screen resolution, offering a consistent experience across web browsers, mobile devices, and tablets. It renders dashboards, scheduling forms, billing summaries, compliance reports, and account management pages dynamically, based on back-end data. The display module 304 ensures that the information shown to users is accurate, relevant, and formatted for intuitive navigation.

Integrated with real-time feedback mechanisms, the display module 304 also presents alerts, status updates, and confirmations that correspond to ongoing transactions. For example, once an insurance verification is completed, the module updates the patient's view with copayment details and scheduling options. Likewise, service providers may see real-time claim status updates and payment timelines directly in their dashboard. The display module also handles user prompts for uploading documents, accepting terms, and confirming appointments or services.

The patient account registration module 311 enables individuals to create and manage their accounts on the MSPS. During registration, users are prompted to enter essential information such as full name, address, contact information, date of birth, insurance provider details, policy numbers, and preferred payment methods. The module includes security layers to authenticate the user's identity and verify that the insurance information provided is valid and linked to an eligible policyholder. Patients may also be required to input demographic details necessary for regulatory reporting or insurer classification.

Once registration is complete, the patient's profile is created and securely stored within the MSPS database. This profile is referenced for all subsequent transactions, including appointment scheduling, copayment processing, and insurance claim submission. Patients may return at any time to update their contact or insurance details, manage notification settings, or add alternative payment methods. The patient account registration module 311 ensures that all data collected during account creation is accessible by the relevant modules while being protected against unauthorized access.

The service provider registration module 313 allows healthcare professionals and transportation service providers to onboard into the MSPS. This includes uploading relevant credentials such as National Provider Identifier (NPI), Tax Identification Number (TIN), state licenses, facility certifications, and insurance documents. Providers may also input their availability, service categories, pricing structures, and geographic service areas. The module ensures that only verified and credentialed service providers are permitted to receive scheduling requests and submit insurance claims.

The registration module also accommodates different provider types including individual practitioners, clinics, non-emergency medical transportation companies, and institutional facilities. Each provider is assigned a unique identifier, and their credentials are indexed for future retrieval by the compliance access control module. The data entered in this module feeds into other parts of the system, such as the display module and scheduling engine, to present accurate and current provider options to patients during booking.

The insurance company registration module 315 is designed to enable private insurers and government insurance programs such as Medicare and Medicaid to establish secure accounts within the MSPS. During registration, insurance entities provide institutional information including company name, payer ID, National Provider Identifier (NPI), address, contact data, and supported plan types (e.g., HMO, PPO, POS, EPO). This data is validated through preconfigured APIs or administrative review to ensure that the entity is a legitimate payer within the healthcare ecosystem. Once approved, the insurance company gains access to compliance and claim audit tools as governed by the compliance access control module.

Each registered insurance company is linked to all claim transactions in which it is the listed payer, enabling the MSPS to associate services, patients, and providers with the insurer's unique identifiers. The insurance company registration module 315 also enables the configuration of data access permissions and regulatory audit capabilities. The module plays a foundational role in enabling real-time benefit verification and post-payment compliance visibility.

The vehicle and driver registration module 317 is specifically tailored to service providers that offer non-emergency medical transportation (NEMT). Through this module, providers input details such as vehicle type (e.g., wheelchair van, ambulance), license plate number, VIN, registration status, insurance documentation, and photos of safety equipment (e.g., fire extinguishers). Simultaneously, drivers are registered by submitting personal identifiers, background checks, driving histories, CPR certifications, and any applicable commercial driver's licenses (CDLs). Each vehicle and driver is issued a unique internal ID for traceability across the MSPS platform.

This module ensures that only verified and compliant vehicles and drivers are used to transport patients whose services will be billed to insurers or paid directly by patients. The registration data captured here supports claim creation, scheduling optimization, and insurer audit trails. The MSPS can restrict transportation bookings to only those drivers and vehicles registered and in good standing in this module, thereby enforcing regulatory compliance and service safety.

The facility/practitioner credential upload interface 319 allows medical professionals and institutions to upload supporting documentation required for system registration, compliance verification, and payer access. This interface accepts scanned copies or digital files of licenses, board certifications, proof of education, DEA registrations, facility permits, business licenses, and malpractice insurance. Each uploaded document is indexed to the provider's account and associated with their unique system ID for retrieval and review.

These credentials may be made visible to insurance companies or regulators through the compliance access control module, depending on user-configured permissions. The MSPS may also flag expired or missing documentation and notify the provider accordingly. This interface ensures that providers remain eligible to offer services through the system and that payers have verifiable documentation in the event of audits or disputes.

The payment method setup module 321 allows both patients and service providers to configure, verify, and manage financial instruments for initiating or receiving payments through the MSPS. Supported payment types include credit and debit cards, ACH bank transfers, digital wallets (e.g., Apple Pay, Google Pay), prepaid vouchers, QR code payment systems, and installment financing platforms. The module supports entry of multiple payment methods and enables users to select default preferences based on transaction type.

The module performs verification of account validity through secure tokenization and PCI-compliant services. For service providers, this module also governs how and where disbursements are sent, particularly in scenarios involving immediate discounted payments. The data stored in this module is encrypted and securely linked to the user's profile to be retrieved automatically during copayment or payout transactions.

The copayment authorization module 323 calculates the patient's copayment responsibility based on the insurance verification response received from the Healthcare Software/Analytics Company System (HSACS). Once calculated, the module prompts the patient for authorization and displays available payment methods configured via the payment method setup module. If the copayment is declined or fails, the system may prompt the patient to try alternate payment methods or reschedule the appointment.

The copayment authorization module ensures that no appointment is finalized unless financial responsibility is confirmed, thereby reducing the risk of claim denial or non-payment. The module logs all user actions and authorization timestamps for audit purposes. It integrates directly with the payment processing module 325 to initiate the financial transaction once authorization is granted.

The payment processing module 325 is responsible for executing all financial transactions through an integrated Payment Gateway System (PGS). This module processes copayments, alternative patient payments, and service provider disbursements. It handles transaction authentication, tokenization, error handling, and secure transfer of payment data. Once a transaction is successfully completed, confirmation is returned to the user and logged into the MSPS database.

The module also supports refund requests, installment tracking, and digital receipt generation. Service providers can configure the module to request immediate payment at a discounted rate upon claim submission. The system uses standardized financial APIs to facilitate interoperability with multiple payment networks.

The appointment confirmation module 327 generates and delivers scheduling confirmations to the patient and service provider following successful completion of the scheduling, verification, and payment steps. The confirmation includes details such as service type, date, time, location, provider name, and payment summary. Notifications may be sent via email, SMS, in-app alerts, or push notifications depending on user preferences.

The module works closely with the display module and the communication module to ensure timely and accurate delivery of confirmations. Each confirmation is stored in the system for future reference, cancellation management, and dispute resolution. The module also triggers synchronization across calendar systems or provider scheduling dashboards where integrated.

The service code generation module 329 is responsible for identifying and assigning appropriate billing codes—such as CPT, STC, ICD-11, HCPCS, and POS codes—based on the nature of the service provided. When a user inputs the service description or selects from a dropdown, the module suggests one or more matching codes and adds them to the patient's transaction record. This ensures that claims are generated in compliance with payer requirements and reduces the chance of denial due to improper coding.

The module also includes logic for detecting mutually exclusive codes and suggesting alternatives based on common combinations for the given service category. Additionally, it supports claim adjustment group code generation to indicate reasons for denial, partial payment, or service modifiers. All generated codes are stored in a structured format and linked to the eventual claim submission.

The claim submission module 331 formats and transmits electronic claims to the Healthcare Software/Analytics Company System (HSACS) upon service completion and input of the Service Completion Code (SCC). The claim includes the patient's insurance data, the provider's credentials, the appointment metadata, and the codes generated by the service code generation module. The module formats the data in accordance with EDI or JSON-based standards accepted by HSACS or third-party clearinghouses.

It also handles acknowledgment receipts, response parsing, and conditional resubmission if required. Failed claims trigger system alerts and error messages that are presented to the service provider for resolution. The module maintains a ledger of all claims and their processing statuses, enabling traceability and compliance tracking.

The claim status feedback module 333 receives claim processing updates from the HSACS and renders them in the patient and provider dashboards. Updates may include claim accepted, pending, rejected, adjusted, or denied. The module provides details on reason codes, payment amounts, and correction requirements if applicable. Visual indicators, such as color-coded statuses and icons, may be used for user clarity.

This module plays a critical role in closing the feedback loop after claim submission. By providing near-real-time updates, it enables timely resubmission, appeals, or alternate billing if necessary. All status updates are logged with timestamps and displayed through the display module for user visibility.

The compliance access control module 335 governs how, when, and by whom transaction and credential data may be accessed for regulatory or insurance purposes. Insurance companies use this module to retrieve service history, verify provider credentials, audit patient appointments, and confirm billing details. Patients and providers can grant or restrict access to specific categories of data such as treatment codes, identity information, or credential documents.

Access permissions are configured at the account level and can be adjusted as needed. Every access attempt is logged with a timestamp, requesting party, and data category. The module ensures compliance with privacy regulations (e.g., HIPAA) and supports audit preparation by maintaining immutable logs of all data views and downloads.

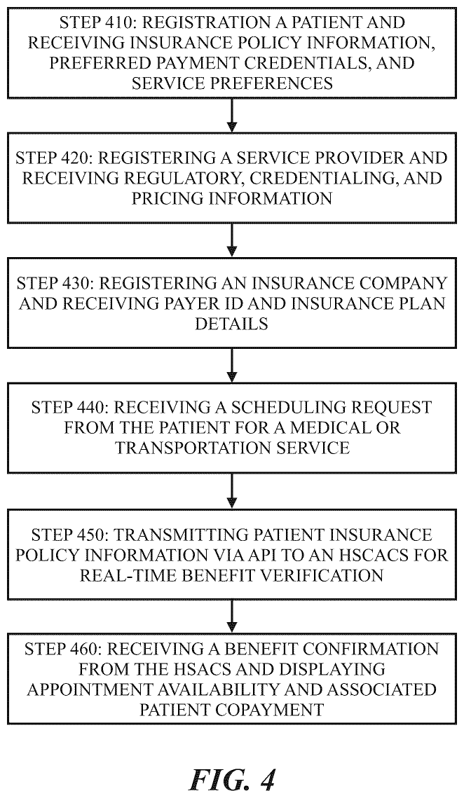

FIG. 4 is a flowchart illustrating a method for facilitating medical service scheduling and payment processing using a Master Service Provider System (MSPS). The illustrated method begins with patient registration and proceeds through a sequence of integrated steps that include service provider and insurance company onboarding, appointment scheduling, real-time benefit verification, and confirmation of copayment obligations. Each step in the method corresponds to a functional process within the MSPS, enabling automated coordination among patients, service providers, and insurance companies. The method emphasizes real-time data exchange, eligibility verification, and transaction transparency to eliminate delays and inefficiencies traditionally associated with medical scheduling and billing. As shown, the MSPS acts as the central platform through which all parties interact, with system-driven workflows ensuring compliance, auditability, and seamless user experiences from initiation through confirmation.

Step 410 initiates the method by enabling a patient to register an account within the Master Service Provider System (MSPS). This registration process is carried out via a patient device, which may include a smartphone, tablet, or other internet-capable computing device. The patient is prompted to input personal information including name, date of birth, and contact details. Critically, the patient must also enter or upload insurance policy information, which may include the insurance provider's name, payer ID, policy number, group number, and plan type (e.g., HMO, PPO, POS, or EPO).

In addition to insurance information, the system collects the patient's preferred payment credentials, such as debit or credit card information, bank account details, digital wallet credentials, or prepaid voucher numbers. These payment credentials are securely tokenized and stored in a protected portion of the patient's account profile. The patient may designate a default payment method for use in future transactions. The patient also specifies service preferences, including the type of service sought (e.g., cardiology consultation, non-emergency transport), appointment time windows, and mobility or accessibility requirements. This data is stored and linked to the patient's profile for future scheduling and billing actions.

Step 420 involves the registration of a service provider using the MSPS platform. A service provider may include a medical professional, a medical facility, a transportation company, or any authorized healthcare service entity. Upon accessing the MSPS through a service provider device, the provider is prompted to input identifying information, including their business or entity name, National Provider Identifier (NPI), Tax Identification Number (TIN), and contact information. The system then requires the upload of credentialing documents such as professional licenses, board certifications, insurance policies, and regulatory permits.

In addition to credentialing data, the service provider inputs service-specific data such as categories of treatment or transport offered, standard procedure codes (e.g., CPT, STC, ICD-11, HCPCS), and pricing information. Pricing may be tiered based on service type, provider specialty, travel distance, wait time, or additional equipment required. The MSPS validates all uploaded documents and stores the information in a provider-specific record, which can be referenced by patients during appointment selection and by insurance companies during post-service audits. The provider also configures availability schedules, which are indexed by the MSPS when matching providers to patient requests.

Step 430 addresses the registration of an insurance company or payer entity within the MSPS. Insurance companies access the platform through an insurance company device and register by inputting institutional data, including payer ID, National Provider Identifier (NPI), and company-specific plan types. Plan types may include variations such as HMO, PPO, EPO, POS, and high-deductible plans. The system may also request contact information, compliance officer details, and broker affiliations where applicable.

Once registered, the insurance company inputs or uploads detailed plan data such as covered services, co-pay structures, coverage limits, and preauthorization requirements. The MSPS stores this data and maps it to the payer ID for efficient matching during eligibility verification steps. This registration process enables real-time integration with the Healthcare Software/Analytics Company System (HSACS), which performs benefit verification using the insurer-provided policy rules. The data gathered during this step ensures that the MSPS can assess coverage eligibility accurately during scheduling and claim submission.

In step 440, the patient submits a scheduling request for a medical or transportation service via their registered MSPS account. The request may include details such as the desired appointment date and time, the service type (e.g., physical exam, diagnostic imaging, wheelchair-accessible transport), preferred providers, and location parameters such as ZIP code or travel radius. The MSPS system accesses the patient's stored insurance and payment information and applies search filters to identify available service providers that meet the specified criteria.

Once candidate service providers are identified, the MSPS displays them to the patient alongside provider profiles, credentialing summaries, pricing data, and patient reviews, where applicable. The patient selects a preferred provider and appointment time, and the system generates a scheduling transaction ID linked to the request. In parallel, the MSPS prepares to initiate benefit verification based on the insurance data already stored in the patient's profile. This step concludes once the patient submits the appointment request, and the system proceeds to initiate insurance verification and copayment evaluation.

Step 450 begins with the MSPS invoking a secure Application Programming Interface (API) call to the Healthcare Software/Analytics Company System (HSACS). The API transmits structured insurance policy data, including the patient's payer ID, policy number, group number, and planned service codes. These codes may have been auto-selected based on the requested service and mapped using the service code generation module. The HSACS queries the insurance company using the transmitted data and applies the insurer's business rules to determine whether the service is eligible under the policy.

Once processed, the HSACS returns a verification report to the MSPS, confirming eligibility status, benefit limits, copayment amount (if any), and coverage exclusions. If the response indicates a denial or partial eligibility, the MSPS may prompt the patient to select an alternate payment method or reschedule the appointment. If the verification is successful, the copayment is calculated and sent to the copayment authorization module for approval. This step ensures that only eligible services are confirmed for scheduling and that payment expectations are clearly communicated to the patient before the service is rendered.

In step 460, the MSPS receives the benefit confirmation from the HSACS and processes the data for presentation to the patient. This includes parsing copayment obligations, benefit limits, and scheduling parameters confirmed as eligible. The system formats this information and updates the patient's interface via the display module, clearly showing the total copayment amount, insurance-covered portion, and any required consents or disclaimers. The service provider is also notified that the appointment has been tentatively matched pending payment authorization.

If a copayment is required, the system invokes the copayment authorization module, prompting the patient to select or confirm a stored payment method. Upon successful authorization, the MSPS finalizes the appointment and issues confirmation notices to both the patient and service provider. This step ensures transparency for all parties involved, eliminates ambiguity around coverage, and reduces administrative friction prior to the service date. The process concludes with a unique appointment ID being stored for use in later steps such as claim generation and compliance reporting.

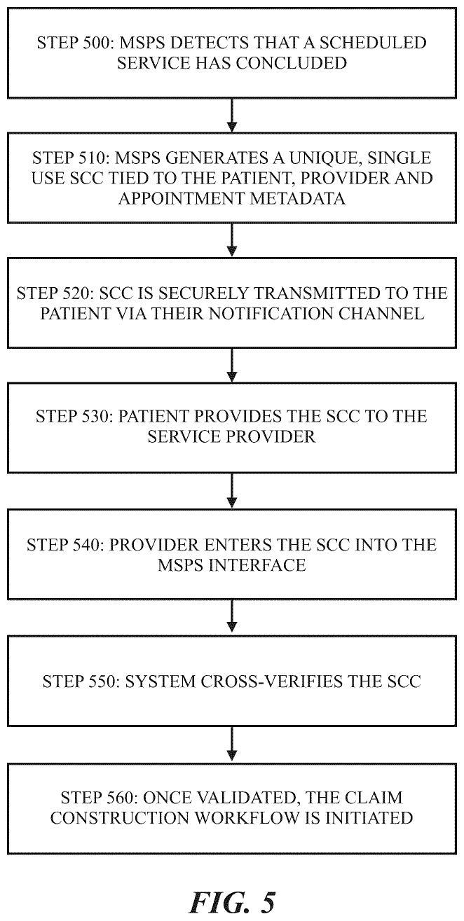

FIG. 5 illustrates the workflow associated with verifying that a service has been completed using a system-generated Service Completion Code (SCC). In step 500, the MSPS detects that a scheduled service has concluded, either through manual provider input or automated system timing. In step 510, the MSPS generates a unique, single-use SCC tied to the patient, provider, and appointment metadata. In step 520, this SCC is securely transmitted to the patient via their preferred notification channel, which may include SMS, email, or push notification.

In step 530, the patient provides the SCC to the service provider as a confirmation of completed service. The provider enters the SCC into the MSPS interface in step 540. The system then cross-verifies the SCC in step 550, ensuring that the code matches the service, appointment ID, and provider credentials. Once validated, step 560 initiates the claim construction workflow. This multi-step SCC process ensures a secure and patient-validated record of service delivery before any claim submission occurs.

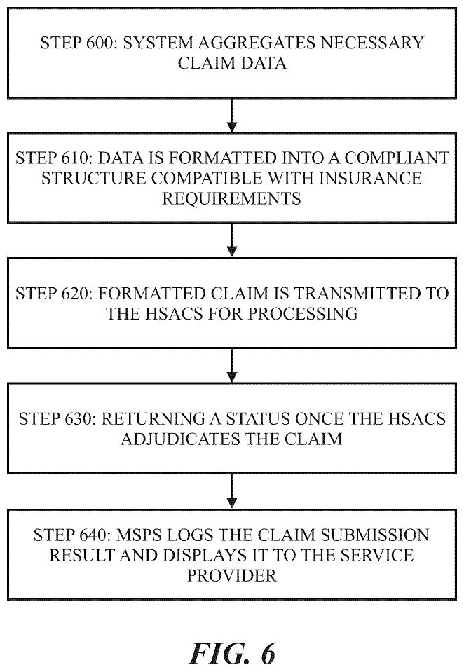

FIG. 6 depicts how the MSPS constructs and transmits a structured insurance claim following SCC validation. In step 600, the system aggregates necessary claim data including standardized billing codes (e.g., CPT, STC, ICD-11, HCPCS), provider credentials, copayment status, and the validated SCC. In step 610, the data is formatted into a compliant structure compatible with insurance requirements, such as EDI 837 or JSON formats for API-based systems.

In step 620, the formatted claim is transmitted to the Healthcare Software/Analytics Company System (HSACS) for processing. The HSACS adjudicates the claim in step 630, returning a status such as accepted, denied, or pending. In step 640, the MSPS logs the claim submission result and displays it to the service provider. This process provides traceability, reduces manual claim preparation, and enables real-time claim monitoring.

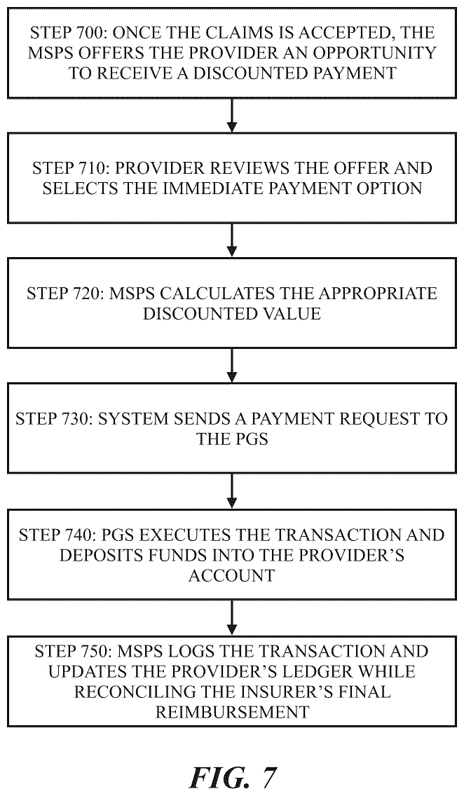

FIG. 7 illustrates the optional immediate payment process offered to service providers upon successful claim submission. In step 700, once the claim has been accepted by HSACS, the MSPS offers the provider an opportunity to receive an immediate discounted payment. In step 710, the provider reviews the offer and selects the immediate payment option if desired. The MSPS calculates the appropriate discounted value in step 720, according to predefined terms agreed upon during provider registration.

In step 730, the system sends a payment request to the Payment Gateway System (PGS), which executes the transaction in step 740, depositing funds directly into the provider's registered account. In step 750, the MSPS logs the transaction and updates the provider's ledger while simultaneously reconciling the insurer's final reimbursement once it is processed. This flow supports cash flow flexibility for providers who prefer quicker payouts over delayed full reimbursements.

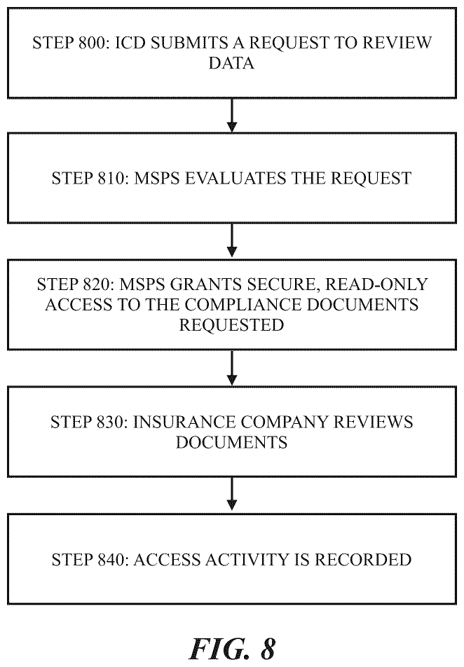

FIG. 8 describes how insurers may access compliance-related documentation following claim payment. In step 800, an Insurance Company Device (ICD) submits a request to review data associated with a completed and paid service. The MSPS evaluates the request in step 810, comparing it against access permissions configured by the patient and service provider. If approved, step 820 allows the MSPS to grant secure, read-only access to the specific compliance documents requested.

In step 830, the insurance company may review documents such as licenses, encounter forms, treatment codes, and proof of service. All access activity is recorded in step 840, capturing the time, user ID, and scope of data viewed. This structured access system helps insurers meet audit and regulatory requirements while preserving patient and provider data control.

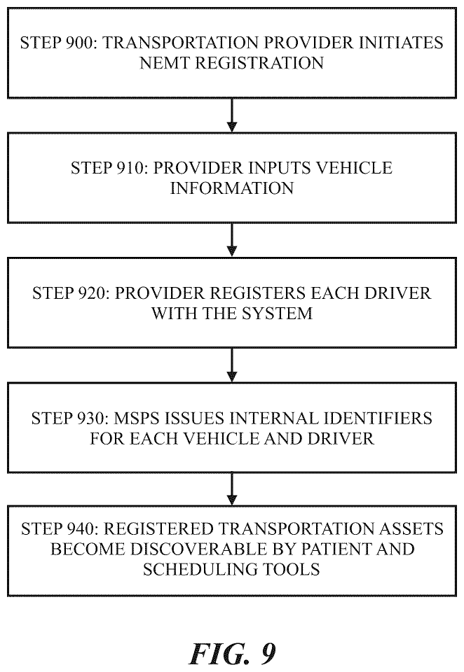

FIG. 9 details how non-emergency medical transportation (NEMT) providers register vehicles and personnel through the MSPS. In step 900, a transportation provider initiates NEMT registration. The provider inputs vehicle information in step 910, including vehicle type (e.g., wheelchair van), registration number, insurance documents, and capacity. In step 920, the provider registers each driver with the system, submitting driver's license, certifications, and background check results.

In step 930, the MSPS issues internal identifiers for each registered vehicle and driver, which are later linked to scheduled transportation services. These identifiers are used to verify compliance and streamline claim generation. In step 940, registered transportation assets become discoverable by patients and scheduling tools based on eligibility and service availability, ensuring regulatory alignment for billed transport claims.

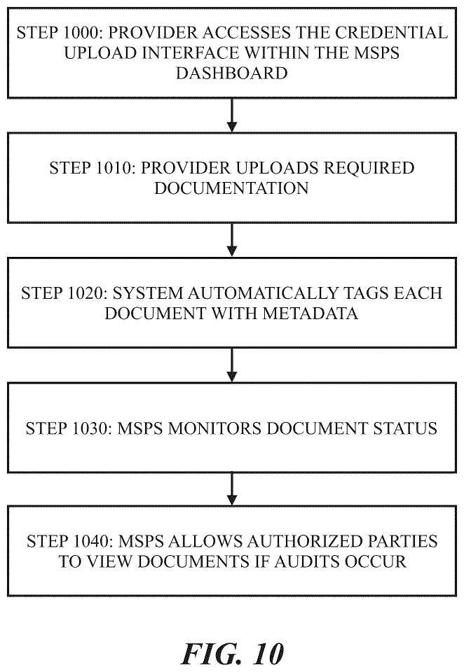

FIG. 10 illustrates how the MSPS manages credential documents uploaded by service providers. In step 1000, a provider accesses the credential upload interface within their MSPS dashboard. In step 1010, the provider uploads required documentation, which may include medical licenses, facility certifications, malpractice insurance, and training records. In step 1020, the system automatically tags each document with metadata such as type, issuing authority, issue and expiration dates.

The MSPS continuously monitors document status in step 1030, sending expiration alerts to the provider when renewals are due. When post-service audits occur or claims are reviewed, the MSPS may allow authorized parties to view documents in step 1040, subject to compliance access control permissions. This process ensures that provider records are current and audit-ready at all times.

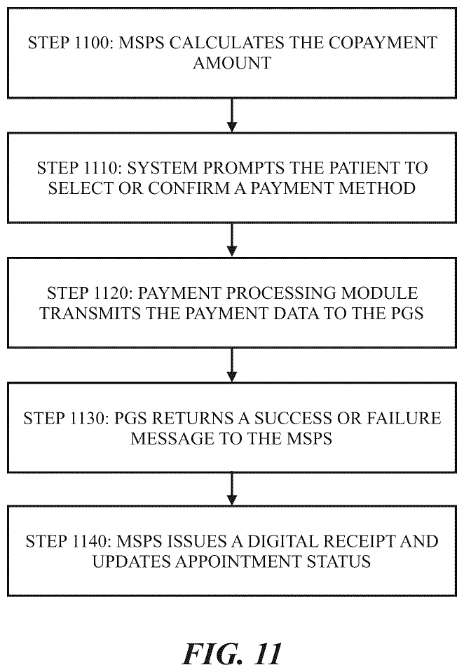

FIG. 11 depicts the process by which copayments are calculated and collected prior to appointment confirmation. In step 1100, the MSPS calculates the copayment amount based on the results of benefit verification returned by the HSACS. In step 1110, the system prompts the patient to select or confirm one of their stored payment methods, such as a credit card, ACH bank account, or digital wallet.

The Payment Processing Module transmits the payment data to the Payment Gateway System in step 1120, where the transaction is executed. In step 1130, the PGS returns a success or failure message to the MSPS. If successful, the MSPS issues a digital receipt and updates appointment status to “confirmed” in step 1140, finalizing the scheduling process. Failed transactions may trigger alternative payment prompts or appointment cancellation.

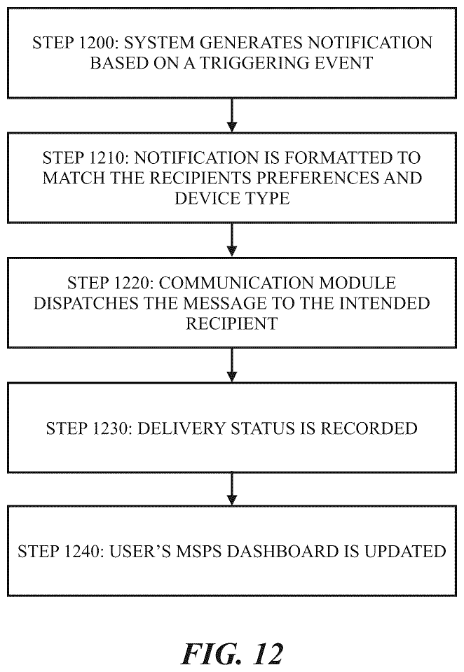

FIG. 12 illustrates the multi-channel alerting and messaging system used by the MSPS to inform stakeholders of status updates throughout the service lifecycle. In step 1200, the system generates a notification based on a triggering event—such as SCC issuance, payment confirmation, claim status update, or credential expiration. In step 1210, the notification is formatted to match the recipient's preferences and device type (e.g., email, SMS, or push notification).

In step 1220, the communication module dispatches the message to the intended recipient, such as a patient, provider, or insurer. Delivery status is recorded in step 1230, which includes success, failure, and read receipt tracking. Finally, in step 1240, the user's MSPS dashboard is updated to reflect the current state of their appointment, claim, or compliance interaction. This ensures real-time transparency and improves user engagement.

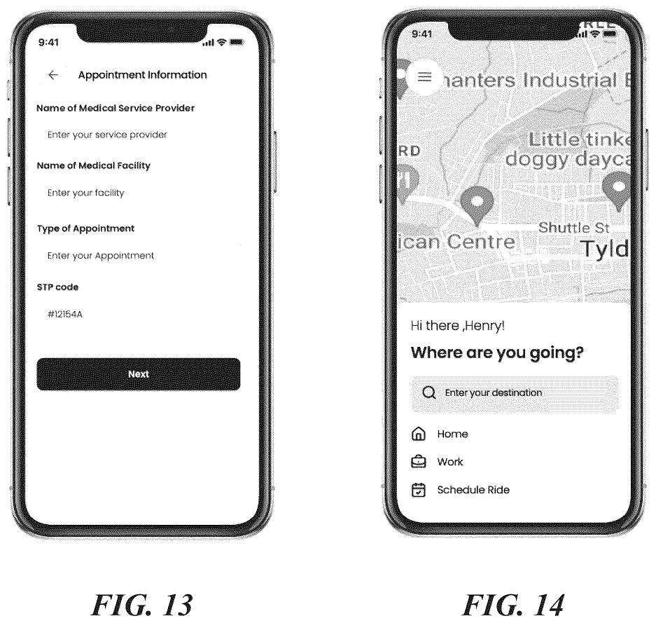

FIG. 13 illustrates a graphical user interface displayed on a patient device showing an appointment information screen generated by the Master Service Provider System (MSPS). The interface includes input fields for the Name of Medical Service Provider, Name of Medical Facility, and Type of Appointment, allowing the patient to identify the specific provider and facility where care will occur. A field labeled STP code allows the patient to input or verify a unique service transaction or scheduling tracking code generated by the MSPS. The Next button at the bottom of the interface allows the user to proceed to subsequent scheduling or verification steps.

The interface is dynamically linked to the MSPS database and validates entries against registered provider and facility records before proceeding. Each field supports auto-completion and data validation features to ensure accurate record association and prevent entry of unregistered provider identifiers. This module promotes data integrity and streamlines the scheduling process by capturing essential information before the insurance verification and claim generation steps occur.

FIG. 14 illustrates a user interface on a patient device that enables a user to enter a destination for a scheduled medical transport or appointment. The display shows an interactive map view integrated with the system's navigation engine, allowing patients to search for a destination address or select from previously saved locations such as Home, Work, or Schedule Ride. The map dynamically updates based on the user's geolocation data, identifying nearby healthcare or transportation facilities.

The destination entry interface communicates directly with the NEMT scheduling subsystem of the MSPS, retrieving provider coverage information and verifying in-network transportation eligibility. This functionality allows patients to plan trips to medical facilities in compliance with insurance parameters and ensures that subsequent vehicle selection options are filtered by eligibility, distance, and time of appointment. The interface thus acts as the initial engagement point in the patient's transportation scheduling workflow.

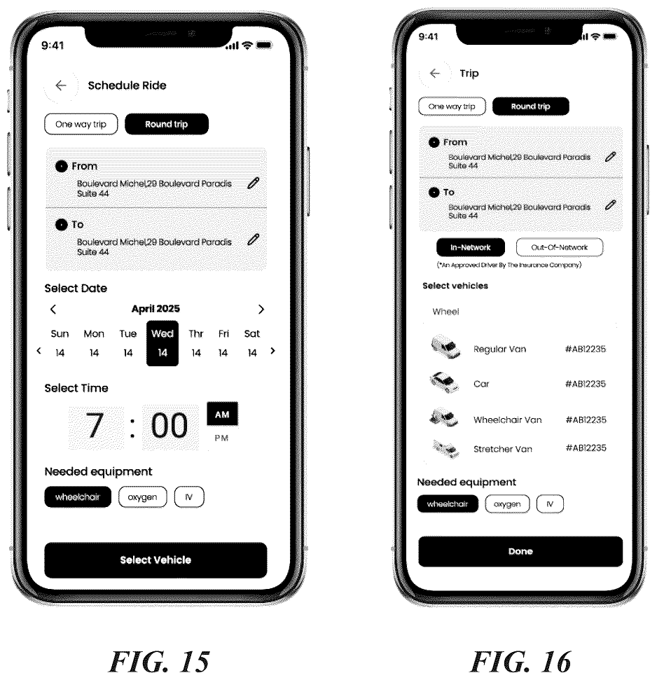

FIG. 15 shows the ride scheduling screen generated by the NEMT module of the MSPS. This interface allows patients to specify trip details including One way or Round trip, pick-up and drop-off locations, and scheduling parameters such as Date and Time. The interface also allows the user to indicate required medical equipment (e.g., wheelchair, oxygen, IV) to ensure the dispatch of properly equipped vehicles. Once the details are entered, the user selects Select Vehicle to proceed.

All input data is transmitted to the MSPS scheduling engine, which verifies resource availability in real-time by matching patient needs with registered NEMT vehicles and drivers. The system also cross-references appointment times with provider schedules and insurance coverage constraints. This design ensures accurate, compliant, and resource-optimized dispatching of transportation assets for medical services.

FIG. 16 illustrates the trip confirmation interface of the NEMT scheduling process. The display includes origin and destination fields, network status indicators (In-Network or Out-of-Network), and a list of selectable vehicles with identifiers and configuration types (e.g., Regular Van, Wheelchair Van, Stretcher Van). The interface also provides an option to indicate required equipment such as wheelchair, oxygen, or IV.

The MSPS dynamically generates this list using verified provider and insurance data, filtering only vehicles and drivers that meet the eligibility, credentialing, and proximity requirements for the patient's scheduled trip. The patient or caregiver may select a preferred vehicle or confirm auto-assignment by the system. Once confirmed, the MSPS locks the vehicle resource and prepares dispatch notifications for both patient and driver.

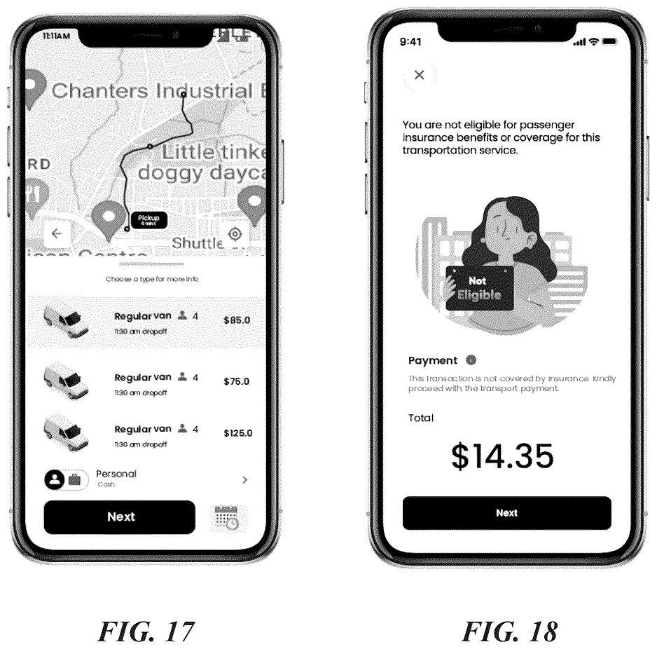

FIG. 17 depicts an interface displaying trip options, pricing, and scheduling information following the confirmation of transportation parameters. The display shows the pickup route overlaid on a map and lists vehicle options with corresponding costs, estimated drop-off times, and passenger capacities. The user can review available options, select a fare, and proceed by selecting Next.

The interface is connected to the Payment Gateway System (PGS) for real-time fare calculation and benefit eligibility checks. The MSPS verifies whether the selected transport qualifies for insurance coverage or whether copayment or direct payment is required. By providing upfront pricing transparency and integrated payment routing, this module reduces administrative overhead and enhances patient experience during transportation scheduling.

FIG. 18 shows a graphical user interface notifying a user of insurance ineligibility for a selected transportation service. The screen displays a visual indicator (e.g., Not Eligible) and text explaining that the transaction is not covered by insurance and that the patient must proceed with out-of-pocket payment. The total cost is clearly presented, along with an action button labeled Next to continue with payment processing.

This interface is generated when the MSPS receives a non-coverage response from the insurance company or when the trip falls outside policy parameters. The system automatically transitions the user to the Payment Method Setup Module, ensuring secure and compliant payment capture through the Payment Gateway System. This workflow maintains continuity of service while preserving compliance with payer rules and patient consent requirements.

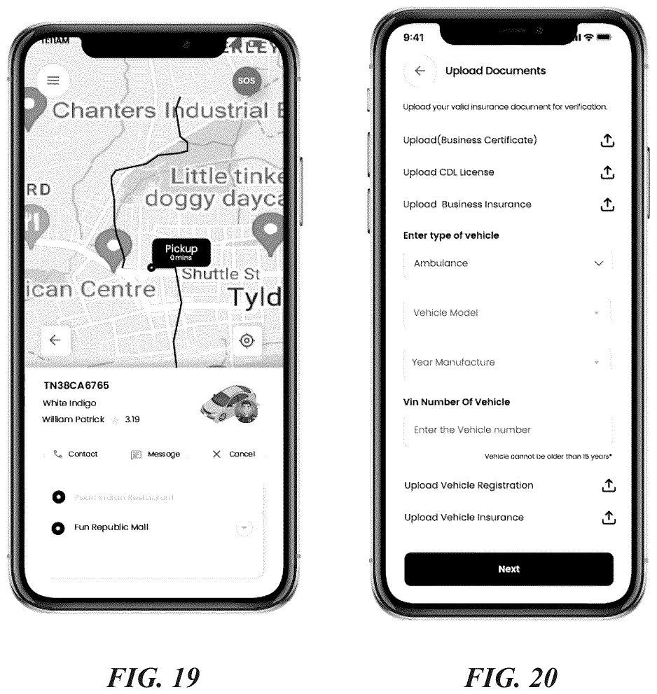

FIG. 19 illustrates a live trip tracking interface that displays the real-time location of the assigned vehicle on a map during patient transport. The screen shows the pickup location, current route, and estimated arrival time. It also includes driver identification information, vehicle details, and direct interaction buttons such as Contact, Message, or Cancel.

This feature is powered by the MSPS's Communication Module, which continuously exchanges telemetry data between the driver's device and the patient's mobile application. The interface provides transparency, safety, and patient confidence by enabling direct, real-time communication and monitoring throughout the journey. Integrated emergency features may allow instant alerts or SOS signaling to dispatch centers or healthcare providers if unexpected events occur.

FIG. 20 illustrates a user interface rendered on a transportation company device for uploading vehicle records and insurance documentation to the Master Service Provider System (MSPS). The interface presents selectable controls to Upload Business Certificate, Upload CDL License, and Upload Business Insurance, followed by structured fields to Enter type of vehicle, Vehicle Model, Year Manufacture, and VIN Number of Vehicle. Example UI text indicates a rule such as “Vehicle cannot be older than 15 years,” which the MSPS enforces during validation. Additional upload controls accept Vehicle Registration and Vehicle Insurance, and a Next control advances the workflow after mandatory items are provided. The layout supports drop-down selectors for constrained entries and text fields for free-form identifiers to streamline data capture and reduce entry errors.

Upon submission, the MSPS associates each uploaded file with the vehicle's unique record and indexes metadata such as document type, issuing authority, effective dates, and expiration dates. Optical character recognition (OCR) and checksum routines may be used to extract and verify VINs and policy numbers, and to detect duplicates or tampered images. The Vehicle and Driver Registration Module 317 and Credential Upload Interface 319 store the artifacts in a provider-scoped repository with access governed by the Compliance Access Control Module 335. Successful validation enables the vehicle to appear as an eligible resource during NEMT scheduling and dispatch, while missing or expired items cause the record to be flagged and excluded until remediation. The MSPS logs each operation for auditability and subsequent insurer review.

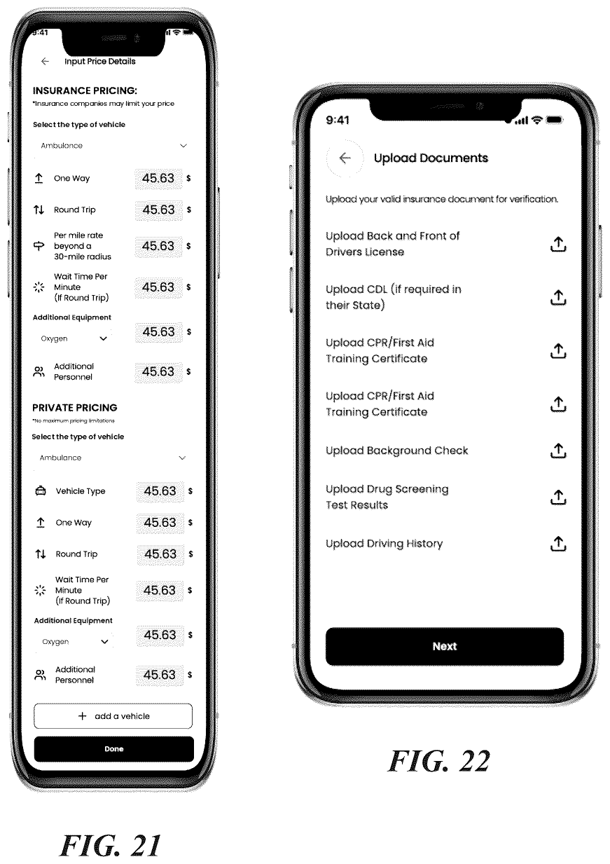

FIG. 21 shows a pricing configuration interface that allows a transportation company to define INSURANCE PRICING and PRIVATE PRICING for a selected type of vehicle. For insurance-covered work, editable fields include One Way, Round Trip, Per mile rate beyond a 30-mile radius, Wait Time per Minute (if Round Trip), Additional Equipment (e.g., oxygen), and Additional Personnel. A notice may state that “insurance companies may limit your price,” indicating payer-defined caps that the MSPS will enforce. A mirrored section labeled PRIVATE PRICING (no maximum pricing limitations) allows the provider to set market rates for self-pay trips using the same categories. Controls such as add a vehicle and Done support multi-vehicle fleet configuration and persistence.

When saved, the MSPS commits the pricing tables to a versioned tariff store keyed by provider, vehicle type, and network status (in-network or out-of-network). The scheduling and quoting engines reference these tables to compute fares, patient copayments, and insurer billable amounts in real time. For insurance pricing, the MSPS applies plan rules obtained during benefit verification to enforce maximums or modifiers and to calculate adjustments such as wait-time caps. All edits are logged with user identity, timestamp, and prior values to support regulatory and contractual audits. The Payment Processing Module 325 and claim assemblers subsequently use these values to populate fare lines and billing codes.

FIG. 22 illustrates a driver-focused upload interface that collects personnel credentials and safety documentation. Upload controls accept Back and Front of Driver's License, CDL (if required in their State), CPR/First Aid Training Certificate, Background Check, Drug Screening Test Results, and Driving History. The interface indicates required items and enables a Next control only when mandated artifacts are present. Icons adjacent to each line provide a consistent action affordance for file selection and status feedback, such as uploaded vs. pending. The layout standardizes document capture across fleets and geographies while supporting state-specific variations.

Once uploaded, the MSPS indexes each file to the driver's unique profile created under Vehicle and Driver Registration Module 317 and stored through Credential Upload Interface 319. Automated validation may include barcode parsing on licenses, expiration checks, and cross-references to sanctioned lists where applicable. The Compliance Access Control Module 335 gates downstream visibility of these files to insurers during audits and to dispatchers during assignment decisions. If any document expires, the system triggers alerts and temporarily prevents new assignments until updates are approved, thereby preserving eligibility and safety standards.

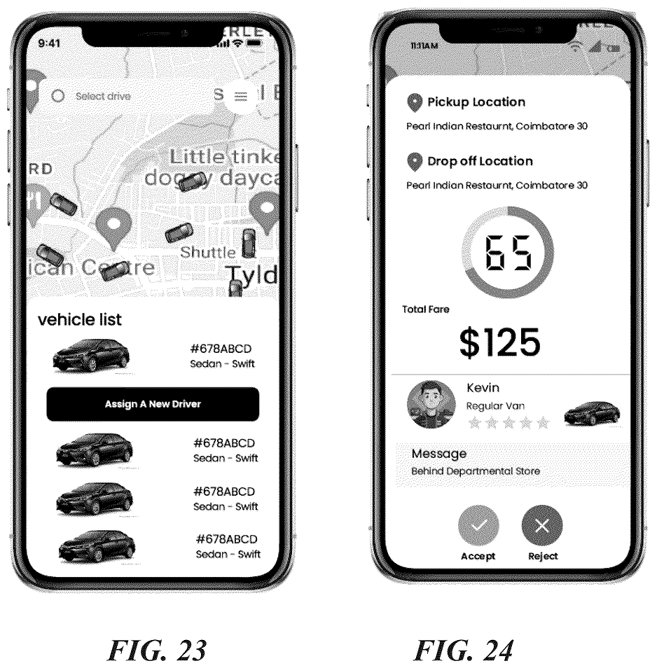

FIG. 23 displays a fleet management interface with a live map and a vehicle list pane showing vehicles and identifiers (e.g., “#678ABCD, Sedan-Swift”). A search control (e.g., Select driver) facilitates locating personnel, and an Assign A New Driver button enables reassignment of a listed vehicle to a different driver. Map icons indicate the current positions of fleet vehicles to aid dispatch decisions and proximity-based matching. The composition provides a single operational view for supervisors to coordinate resources during peak periods or substitutions.

Upon assignment, the MSPS updates relational mappings between driver IDs and vehicle IDs and propagates changes to active scheduling and navigation services. Real-time telematics continue to stream into the MSPS to support ETA computation, route optimization, and accurate mileage capture for billing. Assignment actions are captured with timestamps and user IDs to provide a traceable record for labor, safety, and insurance audits. Eligibility checks based on Module 317 (vehicle/driver compliance) ensure that only credentialed and properly equipped assets are presented for assignment.

FIG. 24 illustrates a job offer interface that presents Pickup Location, Drop off Location, and Total Fare to a driver or dispatcher for decisioning. The interface may show a profile summary (e.g., driver name, vehicle class, rating) and a short Message field with pick-up instructions. A central meter or timer may indicate a response window or offer score. Prominent Accept and Reject controls allow the recipient to finalize the decision.

Upon selection of Accept, the MSPS locks the job to the accepting resource, reserves the route, and pushes confirmations to the patient and service provider. A Reject response can return the job to the queue for the next eligible resource based on proximity, equipment, or load balancing. The system records the decision along with offer time, response time, and outcome for performance analytics and dispute resolution. Accepted jobs immediately synchronize with the navigation screen to begin turn-by-turn guidance.

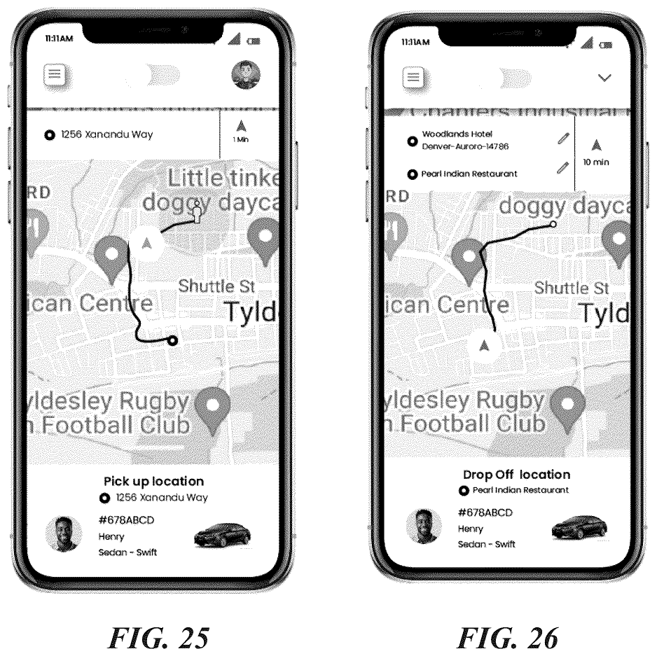

FIG. 25 depicts a navigation interface used while traveling to the pickup location, showing a live route, map markers, and an ETA (e.g., “1 Min”). A banner or list item identifies the pickup address, and a lower panel displays driver identity, vehicle class, and assignment identifier. The UI streamlines mode switching between overview and guidance views, reducing cognitive load during active driving. The map continuously re-centers and recalculates based on traffic and road changes.

Telematics and GPS updates flow through the Communication Module 302 to the MSPS, which simultaneously updates the patient device with ETA and status (“On the way,” “Arriving,” etc.). Arrival detection may be triggered automatically within a geofence or manually by the driver, which transitions the job state to Arrived. These events are logged with timestamps and location coordinates to support accurate time-on-task and subsequent billing or audits. Safety workflows can be invoked when irregular motion or delays are detected.

FIG. 26 shows navigation during transport to the drop-off location, including a highlighted route, destination label, and a time indicator (e.g., “10 min”). The lower panel again identifies the assigned driver, vehicle, and ride identifier for context continuity. The interface may expose quick actions for contacting the passenger or dispatcher if routing changes occur. Dynamic rerouting reacts to closures, traffic density, or care instructions.

Location samples gathered during this phase feed distance, duration, and waypoint metrics into the MSPS for mileage-based pricing and compliance reporting. On approach, the system prepares the Drop-Off Confirmation workflow, which triggers creation of a Service Completion Code (SCC) after confirmation. The captured telematics and timestamps provide defensible evidence of service provision, improving claim acceptance rates. If authorized, insurer portals may later reference these logs during post-payment audits through Module 335.

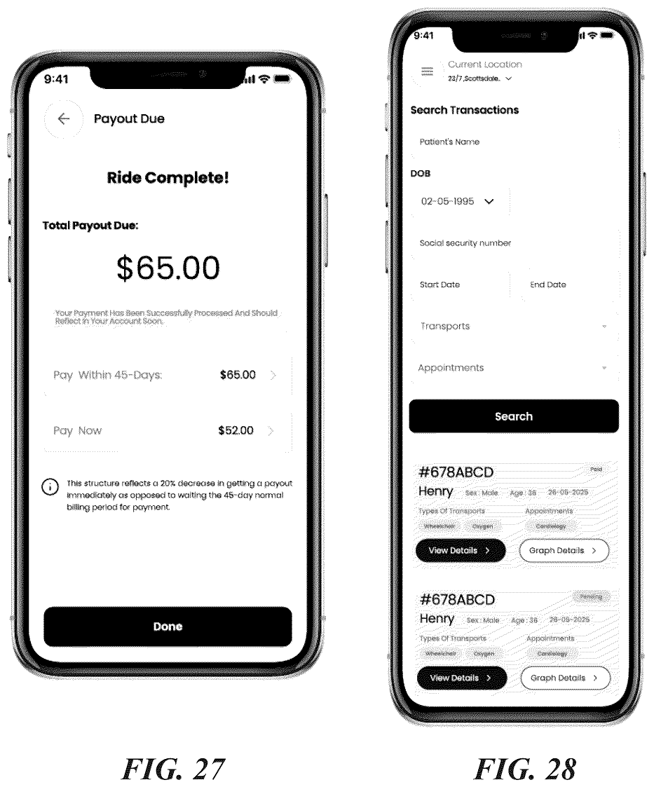

FIG. 27 presents a Payout Due screen following service completion, displaying a Total Payout Due and two selectable disbursement options. A Pay Within 45-Days option reflects the full amount, while a Pay Now option shows a discounted amount (e.g., 20% reduction) for immediate disbursement. Informational text explains the economic tradeoff between accelerated cash flow and standard reimbursement timing. A Done control finalizes the selection once the provider chooses a payout path.

When Pay Now is selected, the Payment Processing Module 325 requests instant settlement from the Payment Gateway System, credits the provider's configured payout account, and records the advance as a factoring event. When Pay Within 45-Days is selected, the MSPS tracks the receivable through the insurer reimbursement cycle and issues payment upon receipt. The ledger reconciles insurer remittances with any prior advances and posts adjustments accordingly. All payouts, selections, and reconciliations are preserved for provider statements and compliance auditing.

FIG. 28 illustrates a consolidated progression interface used by a driver to move between operational states, such as Navigate to Pickup, Picked Up, Navigate to Drop-Off, and Completed. The screen mirrors the map-centric layout but emphasizes state transitions and confirmations required by the MSPS to maintain a reliable audit trail. Each transition can require an explicit tap and may capture timestamps, geofenced confirmations, or optional notes. The interface reduces ambiguity by presenting only context-relevant actions at each stage.

As each state is confirmed, the MSPS updates downstream systems: patient notifications, provider dashboards, and claim pre-fill structures. Completion triggers SCC generation and links recorded distance, time, and pricing parameters to the trip record. If a discrepancy occurs (e.g., wrong destination or missed pickup), the driver can flag the trip for dispatcher review, which pauses claim submission until resolved. This progression ensures that every leg of transport is authenticated and ready for billing and compliance access.

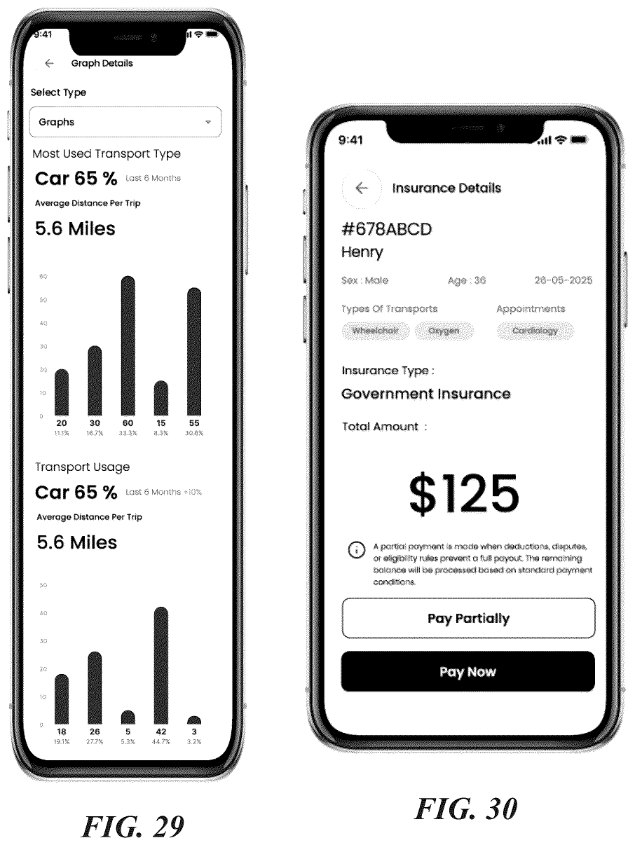

FIG. 29 illustrates a data visualization interface displaying transport analytics for an insured member or provider. The screen includes a Select Type control, enabling the user to toggle between graph types or datasets, such as usage frequency, distance trends, or cost per trip. A section labeled Most Used Transport Type displays a computed ratio (e.g., “Car 65%”) derived from the member's historical transport records over the last six months. The interface also reports Average Distance Per Trip (e.g., “5.6 Miles”) and depicts bar graphs corresponding to various metrics, such as transport counts and frequency distributions. The second portion of the interface repeats these analyses under Transport Usage, presenting comparative statistics and growth indicators (e.g., “+10%”) to highlight utilization changes over time.

The MSPS generates this visualization by aggregating trip and claim data stored in the Transportation Analytics Engine 349, which computes normalized statistics from billing submissions, dispatch logs, and route data. Graphical results are dynamically rendered using data feeds from the Reporting and Insights Module 351, ensuring insurers can monitor efficiency, fraud indicators, and patient transport patterns. Insurers may export this data for compliance reports, provider performance evaluation, or reimbursement audits. The visual representation simplifies anomaly detection, such as overutilization of specific transport types, and aids insurers in validating that the billed services align with the patient's medical necessity and policy terms.

FIG. 30 illustrates a claim settlement interface where the insurance company manages the financial reimbursement of a specific transport transaction. The screen displays the patient's identifying information, trip identifier (e.g., “#678ABCD”), transport types (e.g., Wheelchair, Oxygen), and associated appointment category (e.g., Cardiology). Below this, the interface specifies the Insurance Type (e.g., “Government Insurance”) and the Total Amount billed for the service (e.g., “$125”). Informational text provides context regarding partial payment rules, such as deductions or eligibility adjustments that might prevent a full payout. The insurer can select between Pay Partially or Pay Now controls to initiate a reimbursement through the Claims Settlement Module 347.

When the Pay Now option is selected, the MSPS executes an immediate transaction through the Payment Processing Module 325, updating both the provider and insurer ledgers. Selecting Pay Partially prompts an entry window for specifying the covered amount and generates a pending remainder record for later adjudication. The system maintains audit logs for every payment operation, capturing the claim ID, adjudicator identity, timestamps, and adjustment rationale. Once processed, payment confirmation is transmitted back to the provider interface for reconciliation. This interactive settlement workflow allows insurance representatives to manage reimbursements efficiently while maintaining transparency, compliance, and traceability across all transport-related claims.

In an alternative embodiment, the Master Service Provider System (MSPS) may be configured to support dental care and orthodontic service scheduling in addition to general medical and non-emergency transportation services. Dental patients can use the MSPS to search for available dental providers, input insurance coverage details, and determine eligibility for specific procedures such as cleanings, x-rays, extractions, or orthodontic consultations. The system may include specialized dental CPT and CDT codes integrated into the service code generation module. Claims may be routed through the HSACS for real-time adjudication, and copayments processed in advance of the appointment.

The MSPS may also be deployed within a mental health service delivery model, where patients seek therapy, psychiatric evaluation, or behavioral health support. In this embodiment, therapists, psychologists, and psychiatrists serve as registered service providers, and appointment types may include both in-person and telehealth sessions. The scheduling engine may incorporate additional privacy protocols, such as anonymized patient identifiers or encrypted session notes. Insurance verification may include coverage confirmation for behavioral health benefits, and the payment gateway may support session-based billing or subscription-style care packages.

In another embodiment, the MSPS may be used in rural or underserved healthcare settings where mobile clinics or rotating physicians are common. The system may integrate with mobile health units, enabling patients to schedule appointments with providers visiting on predetermined dates. Transportation scheduling can be automatically tied to mobile clinic availability, ensuring patients without reliable transportation can still receive care. The MSPS can also support government-sponsored insurance programs tailored to rural health initiatives, integrating custom payer IDs and billing rules.

The MSPS may also be adapted for veterinary care, where pet owners use the system to schedule appointments for animal checkups, vaccinations, surgeries, or grooming services. In this use case, the “patient” would be an animal registered under the owner's account, and insurance verification would involve pet insurance carriers. The claim submission module may be modified to comply with veterinary billing codes and animal health compliance regulations. Veterinary clinics can input treatment plans, invoice details, and credentials into the same framework used by human healthcare providers.

An embodiment of the system may extend to school-based health programs, where school nurses, pediatricians, or counselors provide medical and behavioral services to students. The MSPS can be integrated with school databases and configured to track parental consent, vaccination records, and insurance eligibility under Medicaid or CHIP. Scheduling may be initiated by school staff on behalf of the student, and claim submissions routed through state-sponsored clearinghouses. Compliance modules can be configured to generate school-specific reporting formats.

The MSPS can also be implemented within employer-sponsored wellness programs, where employers contract with third-party service providers to offer health screenings, fitness assessments, or chronic condition management services to employees. Employees can access MSPS through workplace portals, schedule appointments, and receive eligibility verification for services covered under employer-funded benefits. Payments may be split between employer funding accounts and employee out-of-pocket contributions, managed through the MSPS's financial workflow. Employers may receive anonymized analytics reports for population health management.

In another alternative embodiment, the MSPS may be tailored for home healthcare services, allowing patients to schedule nursing visits, physical therapy sessions, or in-home lab draws. The system may include modules that account for caregiver credentials, service region restrictions, travel time optimization, and documentation requirements unique to home-based care. Upon service completion, the provider may upload encounter notes, signed patient acknowledgment forms, and photographs of administered medications for claim support. The compliance module may enable auditors or insurance companies to verify documentation tied to home visits.

A further use case envisions the MSPS being adapted for clinical trials and research recruitment, where patients are pre-screened based on eligibility criteria, medical history, and insurance status. Sponsors or research coordinators may register as service providers and publish available trial slots. Patients matching the criteria may schedule screening visits, and the system may track protocol adherence, copay reimbursement, and stipend disbursement. Insurance verification may be limited to ancillary services, while claims tied to trial sponsors may be submitted through research billing entities.

The MSPS may also support group-based or multi-appointment scheduling, allowing facilities to manage family appointments, transportation for multiple patients, or block bookings for recurring treatments such as dialysis or chemotherapy. Patients or caregivers may schedule multiple appointments at once, select the same provider for consecutive sessions, and process a consolidated payment or verification request. Transportation providers may be matched based on vehicle capacity, route efficiency, and specialized equipment. Claim submissions may be bundled or segmented according to payer preference.

In certain embodiments, the MSPS can be offered as a white-labeled platform for state governments, hospital systems, or healthcare exchanges. Organizations may license the MSPS and configure the user interface, logic rules, and data reporting features to reflect their operational policies. Integration with state Medicaid systems or private payer APIs may be pre-built into the white-labeled versions. This embodiment allows scalability of the MSPS model without requiring centralized data management across unrelated health entities.

The system may also be extended for international or cross-border healthcare, where patients seek medical tourism services or second opinions from out-of-country specialists. The MSPS can convert prices to local currency, offer multilingual support, and verify global insurance or self-pay arrangements. Claims may be routed to international insurers or stored for reimbursement after travel. Providers can upload visa support letters, medical summaries, and travel advisories for patient review.

An additional embodiment envisions integration with wearable health monitoring devices. The MSPS can retrieve vital signs or symptom data from smartwatches, glucose monitors, or portable ECGs to automatically suggest appointments, trigger care pathways, or update insurance records. For chronic disease management, the MSPS can generate alerts when a patient's metrics exceed predefined thresholds and recommend real-time scheduling with a relevant provider.

In telemedicine-focused embodiments, the MSPS may facilitate video-based appointments by integrating with secure virtual meeting platforms. Patients can schedule, verify, and pay for remote consultations without visiting a physical location. The claim submission module may be adapted to include telehealth-specific CPT or STC codes and modifiers. Providers may use the credential upload interface to submit telemedicine certifications, and insurers can configure separate verification logic for virtual services.

The MSPS may be configured to serve as a hub for durable medical equipment (DME) ordering and fulfillment, where patients or providers request wheelchairs, oxygen tanks, prosthetics, or other devices. The system verifies coverage, calculates patient responsibility, and schedules delivery or fitting appointments. DME suppliers register like service providers, and claim submissions include equipment codes and possession verification documents. Insurance companies may access compliance data related to DME lifecycle and usage reports.

Lastly, the MSPS may be adapted for non-healthcare service industries with similar scheduling and payment workflows. Examples include legal consultations, educational tutoring, or wellness coaching where credentials, availability, client verification, and secure payments are required. The core structure of the MSPS, modular account registration, scheduling logic, secure API communication, and real-time claim-style reporting, can be repurposed for regulated industries beyond healthcare while maintaining the integrity of the original architecture.

In this disclosure, the various embodiments are described with reference to the flowchart illustrations and/or block diagrams of methods, apparatus (systems), and computer program products.

Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A healthcare transaction system, comprising:
a Master Service Provider System (MSPS) configured to operate on a cloud-based server and accessible via an internet-enabled device;
(or a system of the patient's insurance company) and receive benefit verification and eligibility status;
(or a system of the patient's insurance company) upon receipt of a Service Completion Code (SCC); and
and a compliance access module for granting an insurance company access to service and regulatory data related to a completed and paid-for healthcare event.

2. The system of claim 1, wherein the MSPS further comprises a CPT/STC/ICD-11/HCPCS code-matching engine configured to associate the selected service with one or more standardized medical billing codes.

3. The system of claim 1, wherein the patient interface module includes a geographic service locator configured to identify available service providers based on real-time GPS coordinates of the patient device.

4. The system of claim 1, wherein the payment gateway module accepts payment via credit card, debit card, ACH transfer, digital wallet, prepaid voucher, cryptocurrency wallet, or mobile payment.

5. The system of claim 1, wherein the MSPS transmits a one-time use Service Completion Code (SCC) to the patient upon service completion via text message or email.

6. The system of claim 1, wherein the claim submission module is configured to transmit billing data to the HSAC system (or a system of the patient's insurance company) in real time and receive confirmation of claim acceptance or rejection.

7. The system of claim 1, wherein the MSPS is further configured to issue a discounted immediate payment to the service provider upon confirmation of claim submission and request for early disbursement.

8. The system of claim 1, wherein the compliance access module restricts insurance company access to selected regulatory documents or treatment data based on patient or service provider permissions.

9. The system of claim 1, wherein the MSPS stores all transaction data, including scheduling, billing, insurance verification, and claim outcomes, in a structured, user-specific database.

10. The system of claim 1, wherein the MSPS is configured to enable scheduling for non-emergency medical transportation services based on the patient's mobility needs including stretcher, wheelchair, or ambulatory assistance.

11. A computer-implemented method for facilitating medical service scheduling and payment processing using a Master Service Provider System (MSPS), the method comprising the step of:
registering a patient within the MSPS and receiving insurance policy information, preferred payment credentials, and service preferences;
(or a system of the patient's insurance company) for real-time benefit verification;
and submitting a healthcare claim to the HSAC system (or a system of the patient's insurance company) in real time upon input of the SCC by the service provider.

12. The method of claim 11, further comprising presenting the patient with multiple scheduling options filtered by provider proximity, cost, and service type.

13. The method of claim 11, wherein the MSPS identifies and matches standardized billing codes for each scheduled medical or transportation service based on user inputs.

14. The method of claim 11, wherein the MSPS generates a claim rejection notification and prompts the patient to select an alternative payment method upon eligibility denial.

15. The method of claim 11, further comprising transmitting claim tracking updates from the HSAC system to the patient and service provider in real time.

16. The method of claim 11, wherein the copayment is processed using the patient's default or selected stored payment method.

17. The method of claim 11, wherein the claim is submitted with service provider credentials, license information, and NPI number attached as metadata.

18. A real-time, claim-integrated payment system for medical or transportation services,
the system comprising:
a Master Service Provider System (MSPS) configured to coordinate transactions among a patient, a service provider, an insurance company, a Healthcare Software/Analytics Company (HSAC) (or a system of the patient's insurance company), and a payment gateway;
codes and service data to the HSAC system for immediate claim processing;

19. The system of claim 18, wherein the MSPS calculates the discounted immediate payment based on a preconfigured payment schedule stored in the service provider's account.

20. The system of claim 18, wherein the MSPS provides the patient and insurance company with a downloadable transaction summary including service type, service date, insurance policy applied, and payment confirmation.
